(19) United States
(12) Patent Application Publication
(10) Pub. No.: US 2026/0174409 A1
(43) Pub. Date: Jun. 25, 2026

(54) CALIBRATION DEVICES AND POSITION CALIBRATION METHODS FOR IMAGE-GUIDED SYSTEM, AND CALIBRATION METHODS FOR TREATMENT ROOM POSITIONING SYSTEM

(71) Applicant: MEVION MEDICAL EQUIPMENT CO., LTD., Suzhou (CN)

(72) Inventors: Xiaoyong CHEN, Suzhou (CN); Guihua LI, Suzhou (CN); Chunxu TIAN, Suzhou (CN); Tianxu TANG, Suzhou (CN)

(73) Assignee: MEVION MEDICAL EQUIPMENT CO., LTD., Suzhou (CN)

(21) Appl. No.: 19/533,353

(22) Filed: Feb. 9, 2026

Related U.S. Application Data
(63) Continuation of application No. PCT/CN2024/112037, filed on Aug. 14, 2024.

(30) Foreign Application Priority Data
Aug. 17, 2023 (CN) 202311037022.0
Jan. 19, 2024 (CN) 202410081911.5

(51) Int. Cl. A61B 6/58

(52) U.S. Cl. CPC A61B 6/584; A61B 6/583

(57) ABSTRACT

The present disclosure relates to a calibration device and a position calibration method for an image-guided system, and a calibration method for a treatment room positioning system. The calibration device includes at least two optical path channels, wherein the at least two optical path channels intersect with each other; and at least two imaging assemblies, wherein the two imaging surfaces are disposed at two ends of a same optical channel among the at least two optical path channels; each of the two imaging surfaces is provided with a marking portion, the marking portion is provided with a central marking point, and the central marking point is located in an extending direction of the same one of the at least two optical path channels.

### CROSS-REFERENCE TO RELATED APPLICATIONS

This application is a Continuation of International Application No. PCT/CN2024/112037, filed on Aug. 14, 2024, which claims priority to Chinese Patent Application No. 202311037022.0, filed on Aug. 17, 2023, and Chinese Patent Application No. 202410081911.5, filed on Jan. 19, 2024, the entire contents of each of which are hereby incorporated by reference.

### TECHNICAL FIELD

The present disclosure generally relates to a field of radiation therapy technology, and in particular to a calibration device for an image-guided system, a position calibration method, and a calibration method for a treatment room positioning system.

### BACKGROUND

Radiation therapy is an existing treatment technology, including 2D IGRT (2D Image-Guided Radiation Therapy). The 2D IGRT guides radiotherapy by using two-dimensional images. In the 2D IGRT, a doctor uses an X-ray or other imaging assemblies to obtain two-dimensional images of a corresponding part of a patient, and then uses these images to determine a patient position and a position of a tumor. The information is used to guide positioning and orientation of a radiation source to ensure that the radiotherapy accurately irradiates a tumor region and minimizes damage to surrounding healthy tissues.

In an image-guided radiation therapy system, an isocenter is predetermined, and an intersection point of a pair of X-rays is made to coincide with the isocenter. When radiotherapy is required for a lesion position of a patient, the lesion position of the patient only needs to be moved to the isocenter. Therefore, before performing radiotherapy, a doctor or other equipment user first performs image anatomical feature enhancement processing on an obtained X-ray digital image and a digitally reconstructed radiograph (DRR) generated from a computed tomography (CT) image, and then calculates an offset between a lesion position of the patient and the isocenter through two-dimensional-three-dimensional image registration, thereby adjusting patient positioning by moving a treatment couch before treatment to achieve precise positioning of the tumor.

An offset value between a lesion position of the patient and the isocenter is obtained by performing registration calculation on an obtained pair of X-ray images and a CT image. A pair of X-ray images are related to positions of imaging assemblies, a distance from a ray source to a ray detector, and an angle. A position error of an imaging part may cause an offset error of an X-ray image. For example, if an intersection point of a pair of X-rays does not coincide with the isocenter, or if a pair of X-rays are in an imaging device, calculation of an offset value of a lesion position of the patient may be affected.

Therefore, there is an urgent need for a calibration device for an image-guided system, a position calibration method, and a calibration method for a treatment room positioning system to accurately adjust a positional offset of components in the image-guided system and reduce a position error of an imaging device.

### SUMMARY

One or more embodiments of the present disclosure provide a calibration device for an image-guided system. The calibration device includes at least two optical path channels, wherein each of the at least two optical path channels penetrates the calibration device, and the at least two optical path channels are configured to allow rays to pass through; the at least two optical path channels intersect with each other, and an intersection point of the at least two optical path channels is a center point of the calibration device; at least two imaging assemblies, wherein each of the at least two imaging assemblies includes two imaging surfaces, and the two imaging surfaces are disposed at two ends of a same optical channel among the at least two optical path channels; each of the two imaging surfaces is provided with a marking portion, the marking portion is provided with a central marking point, and the central marking point is located in an extending direction of the same one of the at least two optical path channels; a connection line between the central marking points of the two imaging surfaces in each of the at least two imaging assemblies passes through the center point of the calibration device.

One or more embodiments of the present disclosure provide an image-guided system. The image-guided system includes the calibration device; at least one pair of ray generating devices, configured to generate the rays passing through the at least two optical path channels of the calibration device, wherein rays generated by the at least one pair of ray generating devices intersect with each other; at least one pair of image processing devices, configured to receive the rays passing through the at least two optical path channels of the calibration device and generate image information.

One or more embodiments of the present disclosure provide a position calibration method for an image-guided system. The method includes installing the calibration device to the image-guided system, and aligning the center point of the calibration device with a target point of the image-guided system; emitting, by the at least one pair of ray generating devices, the rays, wherein the rays pass through the calibration device and are received by the at least one pair of image processing devices, and the at least one pair of image processing devices generate the image information of the received rays; transmitting, by the at least one pair of image processing devices, the image information to a data processing device, and obtaining, by the data processing device, positional offset information of at least one of the at least one pair of ray generating devices or the at least one pair of image processing devices based on the image information; adjusting a position of at least one of the at least one pair of ray generating devices or the at least one pair of image processing devices based on the positional offset information; until the position of at least one of the at least one pair of ray generating devices or the at least one pair of image processing devices is adjusted to a designated position.

One or more embodiments of the present disclosure provide a calibration method for a treatment room positioning system. The treatment room positioning system is configured to assist in positioning a target region in a treatment system. The calibration method includes installing a plurality of laser lamps in the treatment room; installing a mechanical isocenter jig on a treatment head, wherein the mechanical isocenter jig is preset with a central reference object, and the central reference object is configured as a spatial physical representation of an isocenter position of the treatment system to configure the treatment system; installing a laser beam verification phantom on the central reference object, telescoping the treatment head, and moving an isocenter position of the laser beam verification phantom to coincide with the isocenter position of the treatment system; wherein the laser beam verification phantom includes three mutually perpendicular surfaces, each surface is preset with a positioning mark and a reflective section opposite to each laser lamp of the plurality of laser lamps, and the positioning mark is adapted to a shape of a laser beam emitted by each laser lamp; the laser beam verification phantom is provided with a loading hole, a virtual connection line between the isocenter position of the laser beam verification phantom and the loading hole extends to a central position of the central reference object, and the virtual connection line between the isocenter position of the laser beam verification phantom and the loading hole is configured to be coaxial with a telescopic path of the treatment head; activating each laser lamp, so that each laser lamp emits a laser beam toward the laser beam verification phantom along three mutually perpendicular directions, respectively; determining whether the laser beam emitted by each laser lamp coincides with the positioning mark of the laser beam verification phantom; and whether a laser beam path reflected by the reflective section coincides with the laser beam path emitted by each laser lamp; if the laser beam emitted by each laser lamp coincides with the positioning mark of the laser beam verification phantom; and a laser beam path reflected by the reflective section coincides with the laser beam path emitted by each laser lamp, not adjusting a laser lamp position of the each laser lamp; if one of the laser beam emitted by each laser lamp does not coincides with the positioning mark of the laser beam verification phantom; and a laser beam path reflected by the reflective section does not coincides with the laser beam path emitted by each laser lamp, adjusting the laser lamp position of each laser lamp, so that the laser beam emitted by the each laser lamp coincides with the positioning mark of the laser beam verification phantom, and the laser beam path reflected by the reflective section coincides with the laser beam path emitted by each laser lamp.

One or more embodiments of the present disclosure provide a treatment room positioning system. The treatment room positioning system is configured to execute the calibration method. The treatment room positioning system includes a wall body, preset with a mounting position; the plurality of laser lamps, installed at the mounting position of the wall body; and the laser beam verification phantom, wherein the isocenter position of the laser beam verification phantom is located at an intersection point of laser beams emitted by the plurality of laser lamps.

### BRIEF DESCRIPTION OF THE DRAWINGS

FIG. 1 is a schematic structural diagram of an image-guided system according to some embodiments of the present disclosure.

FIG. 2 is a schematic structural diagram of a calibration device according to some embodiments of the present disclosure.

FIG. 3 is a schematic planar cross-sectional view of a calibration device according to some embodiments of the present disclosure.

FIG. 4 is a schematic structural diagram of an imaging surface according to some embodiments of the present disclosure.

FIG. 5 is a schematic structural diagram of a positioning surface according to some embodiments of the present disclosure.

FIG. 6 is a schematic diagram of partial ray irradiation paths when a ray generating device is offset according to some embodiments of the present disclosure.

FIG. 7 is a schematic diagram of image information obtained by partial image processing devices when a ray generating device is offset according to some embodiments of the present disclosure.

FIG. 8 is a schematic diagram of partial ray irradiation paths when an image processing device is offset according to some embodiments of the present disclosure.

FIG. 9 is a schematic diagram of a connection of a control device, a ray generating device, and an image processing device according to some embodiments of the present disclosure.

FIG. 10 is a schematic structural diagram of a treatment room positioning system installed in a treatment room according to some embodiments of the present disclosure.

FIG. 11 is a schematic structural diagram of another perspective when a treatment room positioning system is installed in a treatment room according to some embodiments of the present disclosure.

FIG. 12 is a partial exploded view of a wall body and a laser lamp in a treatment room positioning system according to some embodiments of the present disclosure.

FIG. 13 is a schematic diagram of a structure when a treatment head, a mechanical isocenter jig, and a laser beam verification phantom are connected according to some embodiments of the present disclosure.

FIG. 14 is an exploded schematic diagram of a mechanical isocenter jig and a laser beam verification phantom according to some embodiments of the present disclosure.

FIG. 15 is a cross-sectional view of a laser beam verification phantom according to some embodiments of the present disclosure.

Reference numerals: 100, calibration device; 1, optical path channel; 2, imaging assembly; 21, imaging surface; 211, marking portion; 3, recessed groove; 4, light-transmitting hole; 5, positioning channel; 6, positioning assembly; 61, positioning surface; 611, positioning portion; 7, mounting hole; 8, calibration hole; 9, calibration sphere; 200, ray generating device; 300, image processing device; 500, control device; N, central positioning point; M, central marking point; P, center point; A, B, coordinate point; C, first endpoint; D, second endpoint; 401, wall body; 4011, mounting slot; 402, laser lamp; 403, laser beam verification phantom; 4031, positioning mark; 4032, reflective section; 4033, loading hole; 4034, positioning metal sphere; 4035, horizontal scale line; 4036, vertical scale line; 404, mechanical isocenter jig; 4041, base; 4042, central reference object; 405, position measuring instrument; 406, treatment head.

### DETAILED DESCRIPTION

Exemplary embodiments will now be described more fully with reference to the accompanying drawings. However, the exemplary embodiments can be implemented in various forms and should not be construed as limited to the embodiments set forth herein. Rather, these embodiments are provided so that the present disclosure is thorough and complete, and fully conveys the concepts of the exemplary embodiments to those skilled in the art. In the drawings, the same reference numerals denote the same or similar structures, and thus repeated descriptions thereof will be omitted.

The terms describing positions and directions in the present disclosure are all explained by way of example with reference to the accompanying drawings, but the terms may be changed as needed, and all such changes are included within the protection scope of the present disclosure.

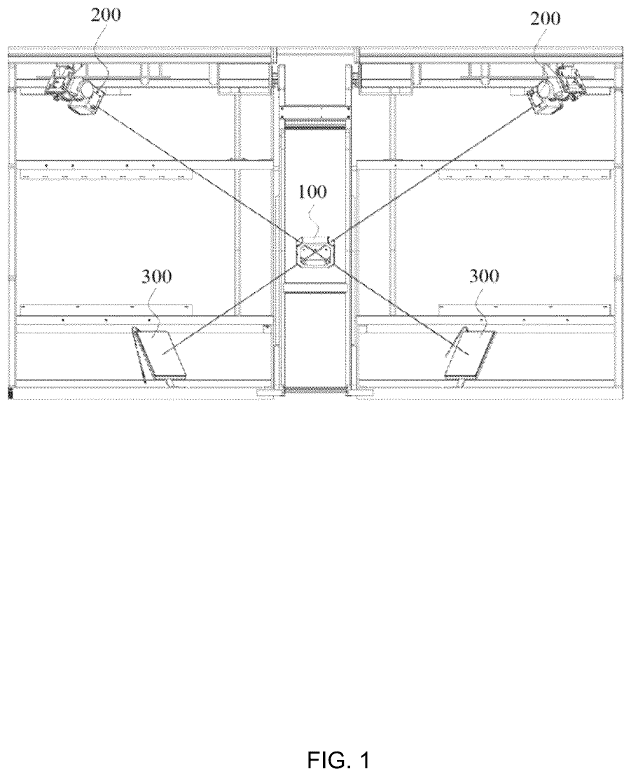

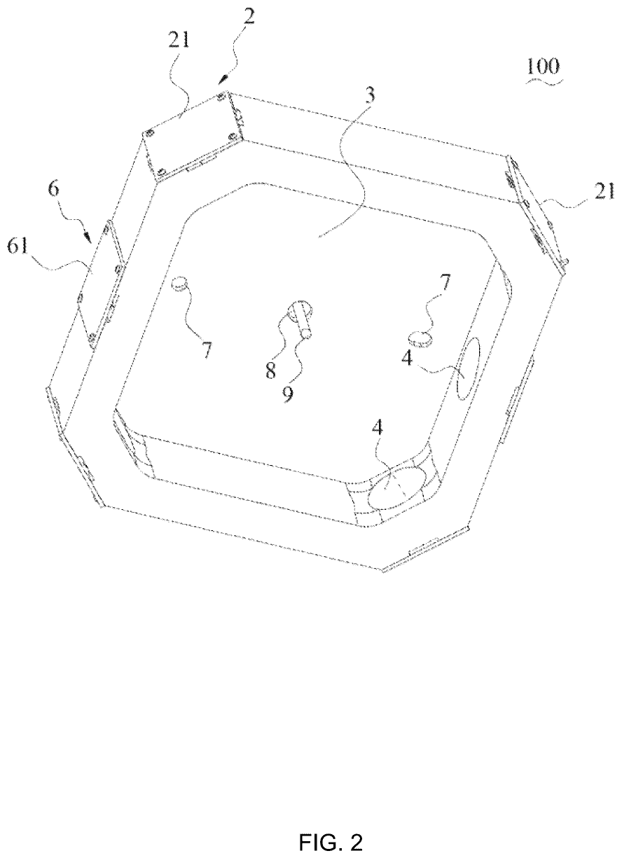

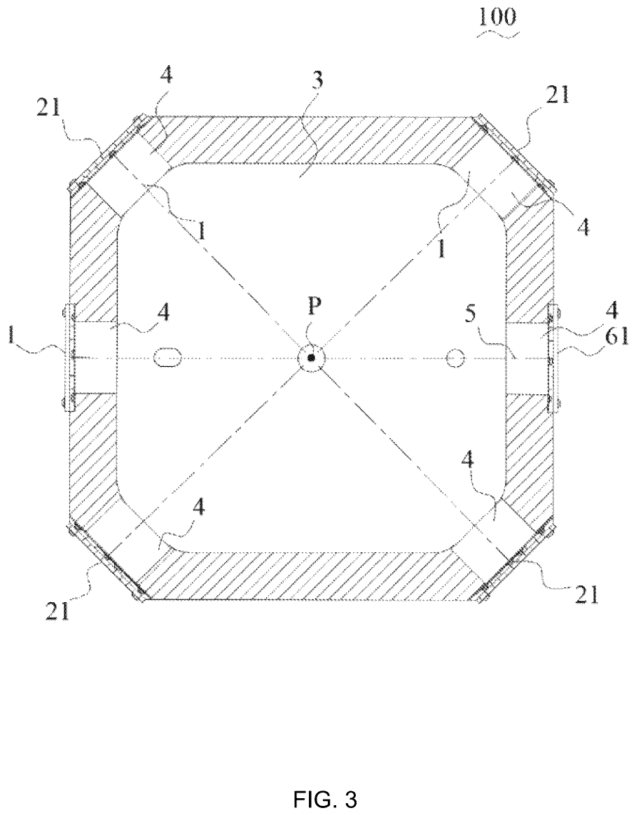

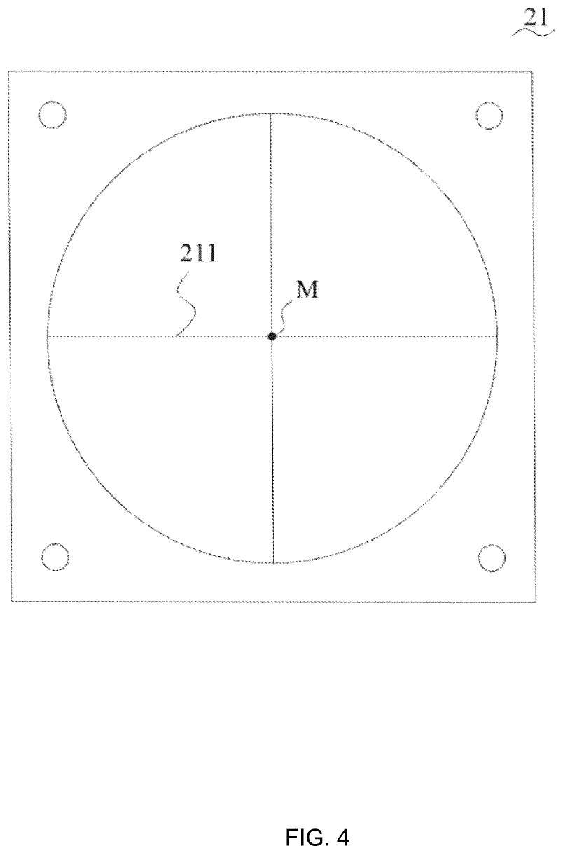

FIG. 1 is a schematic structural diagram of an image-guided system according to some embodiments of the present disclosure. FIG. 2 is a schematic structural diagram of a calibration device according to some embodiments of the present disclosure. FIG. 3 is a schematic planar cross-sectional view of a calibration device according to some embodiments of the present disclosure. FIG. 4 is a schematic structural diagram of an imaging surface according to some embodiments of the present disclosure.

In some embodiments, as shown in FIG. 1 to FIG. 4, a calibration device for an image-guided system (hereinafter referred to as a calibration device 100) includes at least two optical path channels 1, each of the at least two optical path channels 1 penetrates the calibration device 100, and the at least two optical path channels 1 are configured to allow rays to pass through. The at least two optical path channels 1 intersect with each other, and an intersection point of the at least two optical path channels 1 is a center point of the calibration device 100. The calibration device 100 includes at least two imaging assemblies 2, each of the at least two imaging assemblies 2 includes two imaging surfaces 21, and the two imaging surfaces 21 are disposed at two ends of a same optical path channel 1 among the at least two optical path channels 1. Each of the two imaging surfaces 21 is provided with a marking portion 211. The marking portion 211 is provided with a central marking point M. The central marking point M is located in an extending direction of the same one of the at least two optical path channels 1. A connection line between the central marking points M of the two imaging surfaces 21 in each of the at least two imaging assemblies 2 passes through the center point of the calibration device 100.

The calibration device 100 is a device for calibrating a positional error of components of the image-guided system. The image-guided system is a part of a radiotherapy device, and the image-guided system is a system for guiding or directly controlling a treatment couch for accurate positioning.

The optical path channel 1 is a channel for allowing rays (e.g., X-rays) to pass through, so that the rays can pass through the calibration device 100 via the optical path channel 1. Each of the at least two optical path channels 1 penetrates the calibration device 100.

In some embodiments, the calibration device 100 includes at least two optical path channels 1.

As shown in FIG. 2 and FIG. 3, the calibration device 100 includes at least two optical path channels 1 intersecting with each other, and the intersection point of the at least two optical path channels 1 is a center point P of the calibration device 100. The center point P of the calibration device 100 is a geometric center of the calibration device 100.

It should be noted that the count of optical path channels 1 shown in FIG. 2 and FIG. 3 is two, which is merely an example. The count of optical path channels 1 is adaptively set according to requirements, for example, the count of optical path channels 1 is four, etc.

In some embodiments, an optical path channel 1 is formed by a pair of light-transmitting holes 4 and at least a portion of a recessed groove 3. More descriptions regarding the light-transmitting holes 4 and the recessed groove 3 may be found in the related description below.

The imaging assembly 2 is a component for generating a pair of projection images for calibration.

In some embodiments, the calibration device 100 includes at least two imaging assemblies 2.

As shown in FIG. 2, in some embodiments, an imaging assembly 2 includes two imaging surfaces 21.

In some embodiments, the imaging assembly 2 may also include other counts of imaging surfaces 21. In the following, the imaging assembly 2 is described as including two imaging surfaces 21.

The imaging surface 21 is a component for generating one of a pair of projection images for calibration.

As shown in FIG. 4, the two imaging surfaces 21 are disposed at two ends of a same optical path channel 1 among the at least two optical path channels 1, so that when rays pass through the same optical path channel 1, a pair of projection images can be formed on the two imaging surfaces 21 on the optical path channel 1.

The marking portion 211 is a specific pattern on the imaging surface 21, so as to facilitate positioning of a projection image on the imaging surface 21 through the marking, such as scales, etc.

In some embodiments, as shown in FIG. 4, the marking portion 211 is a crosshair structure, and an intersection point of the crosshairs is the central marking point M of the marking portion 211.

In some embodiments, the marking portion 211 is set to other shapes, such as other structures with intersecting lines, an intersection point of the intersecting lines serving as the central marking point M of the marking portion 211; or other structures with a point-like structure, the point-like structure serving as the central marking point M of the marking portion 211.

The central marking point M is an intersection point of an extension direction of an optical path channel 1 and an imaging surface 21.

The extension direction of an optical path channel 1 is a theoretical path direction along which rays pass through the calibration device 100.

In some embodiments, disposing of an imaging surface at each end of an optical path channel enables a ray passing through the optical path channel to form a projection of a pair of imaging surfaces. According to an offset of central marking points of the projections of the pair of imaging surfaces, an offset of the ray generating device and the image processing device in an image-guided system can be determined, thereby facilitating adjustment of positions of the ray generating device and the image processing device.

In some embodiments, the at least two optical path channels 1 are formed by extending along a pair of intersecting diagonals in the calibration device 100; and/or, the two imaging surfaces 21 are disposed at a vertex of the calibration device 100. Merely by way of example, when the calibration device 100 includes an approximately square structure, and each of four corners of the square structure is provided with a cut surface, an optical path channel 1 extends along a diagonal of the calibration device 100. An intersection point of two optical path channels 1 is a center point of the square structure, i.e., a center point P of the calibration device 100. The imaging surfaces 21 are located at the cut surfaces provided at the four corners of the square structure.

In some embodiments, the two optical path channels are respectively disposed along diagonals of the calibration device 100 (assuming the calibration device 100 is a cube or a rectangular parallelepiped). An included angle between the two optical path channels can be maximized (e.g., approaching 90 degrees in a cube). By disposing the imaging surfaces at the vertices, high-sensitivity detection of translational offsets in that direction is achieved, maximizing calibration performance within a limited space.

In some embodiments, the calibration device 100 is provided with a recessed groove 3 and a plurality of light-transmitting holes 4. The center point P of the calibration device 100 is located in the recessed groove 3. One end of each of a plurality of light-transmitting holes 4 penetrates the calibration device 100, and another end of each of the plurality of light-transmitting holes 4 communicates with the recessed groove 3. A pair of light-transmitting holes 4 located on opposite sides of the center point P of the calibration device 100 and the recessed groove 3 form one of the at least two optical path channel 1 together, and the two imaging surfaces 21 block the plurality of light-transmitting holes 4.

The recessed groove 3 is a cavity located inside the calibration device 100, and configured to communicate with the plurality of light-transmitting holes 4 to form a complete optical path channel 1.

The light-transmitting hole 4 is a hole penetrating a wall surface of the calibration device, configured to serve as an entrance and an exit of the optical path channel 1.

In some embodiments, as shown in FIG. 2, one end of a light-transmitting hole 4 penetrates the calibration device 100 (e.g., the one end of the light-transmitting hole 4 opens on the calibration device 100). Another end of the light-transmitting hole 4 communicates with the recessed groove 3 (e.g., the another end of the light-transmitting hole 4 opens on the recessed groove 3), i.e., the calibration device 100 and the recessed groove 3 communicate via the light-transmitting hole 4.

In some embodiments, a pair of light-transmitting holes 4 are located on opposite sides of the center point P. At least a portion of the recessed groove 3 communicates with the pair of light-transmitting holes 4, such that the pair of light-transmitting holes 4 and the portion of the recessed groove 3 connecting the pair of light-transmitting holes 4 form one optical path channel 1, i.e., the pair of light-transmitting holes 4 located on opposite sides of the center point of the calibration device 100 and the recessed groove 3 together form one optical path channel 1 of at least two optical path channels 1.

In some embodiments, the light-transmitting holes 4 are disposed at vertices of the calibration device 100. The two intersecting optical path channels 1 are correspondingly formed by extending along a pair of intersecting diagonals in the calibration device 100. Merely by way of example, when the calibration device 100 has an approximately square structure, and each of four corners of the square structure is provided with a cut surface, four of the plurality of light-transmitting holes 4 are correspondingly disposed at the cut surfaces of the four corners of the square structure. An optical path channel 1 formed by one pair of light-transmitting holes 4 and at least a portion of the recessed groove 3 extends along one diagonal of the calibration device 100. Another optical path channel 1 formed by another pair of light-transmitting holes 4 and at least a portion of the recessed groove 3 extends along another diagonal of the calibration device 100. An intersection point of the two optical path channels 1 is a center point of the square structure, i.e., the center point P of the calibration device 100.

In some embodiments, as shown in FIG. 3, the imaging surface 21 blocks the light-transmitting hole 4.

In some embodiments, as shown in FIG. 3 and FIG. 4, a marking portion 211 of the imaging surface 21 is located above, below, or within the light-transmitting hole 4 along an extension direction of the corresponding optical path channel 1. A periphery of the imaging surface 21 abuts against an outer side wall of the calibration device 100, or the periphery of the imaging surface 21 is fixed to the outer side wall of the calibration device 100. Merely by way of example, the periphery of the imaging surface 21 is fixed to the outer side wall of the calibration device 100 via fasteners such as screws.

In some embodiments, communicating a pair of light-transmitting holes via the recessed groove conveniently forms a complete, penetrating optical path channel, ensuring straight-line propagation of rays. Blocking the light-transmitting holes with the imaging surfaces provides a stable and reliable reference for precise measurement of projection offsets.

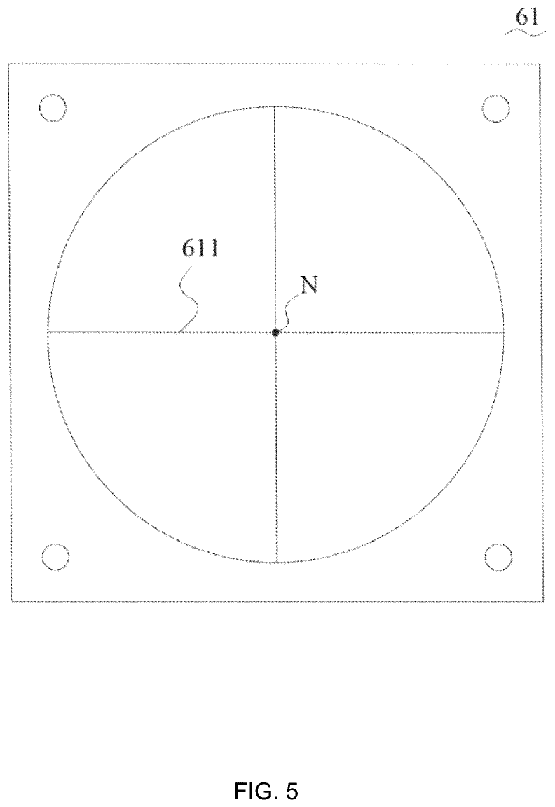

FIG. 5 is a schematic structural diagram of a positioning surface according to some embodiments of the present disclosure.

In some embodiments, as shown in FIG. 2, FIG. 3, and FIG. 5, the calibration device 100 further includes: a calibration sphere 9, configured to indicate an isocenter of the image-guided system, wherein the calibration sphere 9 coincides with the center point of the calibration device 100; a mounting hole 7, configured to fix the calibration device 100 to the image-guided system; a calibration hole 8, configured to install the calibration sphere 9; a positioning channel 5, penetrating the calibration device 100, wherein the positioning channel 5 is configured to allow light to pass through; the positioning channel 5 extends along a center line in a horizontal direction of the calibration device 100, and the positioning channel 5 passes through the center point of the calibration device 100; a positioning assembly 6, including a pair of positioning surfaces 61, wherein the pair of positioning surfaces 61 are disposed at opposite ends of the positioning channel 5, the pair of positioning surfaces 61 are provided with positioning portions 611, the positioning portions 611 are provided with central positioning points N, and a connection line between the central positioning points N of the pair of positioning surfaces 61 passes through the center point of the calibration device 100.

The calibration sphere 9 is a small sphere preset in the image-guided system to coincide with the target point. The target point is the isocenter of the image-guided system, i.e., the calibration sphere 9 is configured to indicate the isocenter of the image-guided system.

In some embodiments, after the calibration device 100 is installed in the image-guided system, the calibration sphere 9 coincides with the center point P of the calibration device 100, for correcting a position of the calibration device 100. A size of the calibration sphere 9 is much smaller than a size of the calibration device 100, so that a user can observe the calibration sphere 9 while the calibration sphere 9 does not excessively block rays passing through the optical path channel 1 or light passing through the positioning channel 5.

The mounting hole is a hole for fixing the calibration device 100 to the image-guided system. Merely by way of example, the mounting hole 7 is used for a screw to pass through, so that the calibration device 100 is installed at a corresponding position in the image-guided system.

In some embodiments, a plurality of mounting holes 7 are provided, e.g., two mounting holes 7 are provided.

The calibration hole 8 is a hole for mounting the calibration sphere 9.

In some embodiments, the calibration hole 8 is configured to fix the calibration sphere 9 and allow the calibration sphere 9 to pass through.

In some embodiments, a connection line between the calibration hole 8 and the target point is parallel to a thickness direction of the calibration device 100.

The positioning channel 5 is a channel penetrating the calibration device 100 and configured to allow light to pass through.

In some embodiments, the positioning channel 5 passes through the center point P of the calibration device 100, and the positioning channel 5 extends along a center line of the calibration device 100. The center line is a center line of the calibration device 100 in a vertical direction or a horizontal direction after the calibration device 100 is installed at a designated position in the image-guided system. Preferably, the positioning channel 5 extends along the center line of the calibration device 100 in the horizontal direction.

In some embodiments, the positioning channel 5 intersects with the optical path channel 1, and an intersection point of the positioning channel 5 and the optical path channel 1 is the center point P of the calibration device 100.

In some embodiments, the positioning channel 5 is formed by a pair of light-transmitting holes 4 and at least a portion of the recessed groove 3. Merely by way of example, a pair of light-transmitting holes 4 are disposed at opposite ends along a center line direction (e.g., a center line in the horizontal direction) of the calibration device 100 relative to the center point P. At least a portion of the recessed groove 3 communicates with this pair of light-transmitting holes 4, and this pair of light-transmitting holes 4 and the at least a portion of the recessed groove 3 form the positioning channel 5. A structure of the light-transmitting holes 4 forming the positioning channel 5 is similar to the structure of the light-transmitting holes 4 forming the optical path channel 1. More descriptions may be found in the related description.

The positioning assembly 6 is a component configured to perform preliminary positioning when the calibration device 100 is mounted on the image-guided system. For example, after the calibration device 100 is installed on the image-guided system, the calibration device 100 is preliminarily positioned by adjusting the calibration device 100 such that a connection line between central positioning points N of a pair of positioning surfaces 61 in the calibration device 100 passes through a target point in the image-guided system.

In some embodiments, as shown in FIG. 5, the positioning assembly 6 includes a pair of positioning surfaces 61. The pair of positioning surfaces 61 are disposed at opposite ends of the positioning channel 5.

In some embodiments, the positioning assembly 6 includes three positioning surfaces 61, or the like. Two of the three positioning surfaces 61 are disposed at opposite ends of the positioning channel 5, and another one of the three positioning surfaces 61 is disposed at any position in the middle of the positioning channel 5. It should be noted that the count of the positioning surfaces 61 shown in FIG. 5 is merely an example. The count of the positioning surfaces 61 may be adaptively set according to requirements.

The positioning surface 61 is a reference surface configured to quickly determine a basic orientation of the calibration device 100 in space. For example, the positioning surface 61 is a coordinate surface with a crosshair scale.

The positioning surface 61 is provided with a positioning portion 611. The positioning portion 611 is provided with a central positioning point N. A connection line between the central positioning points N of the pair of positioning surfaces 61 passes through a center point P of the calibration device 100.

The positioning portion 611 is a reference mark (e.g., a crosshair) engraved on the positioning surface 61. The central positioning point N is a preset reference point (e.g., an intersection point of the crosshair) on the positioning portion 611.

In some embodiments, a shape of the positioning portion 611 is identical to a shape of the marking portion 211.

In some embodiments, the calibration device provides a dual positioning reference through the calibration sphere and the positioning assembly. Coarse adjustment using the positioning assembly and fine adjustment using the calibration sphere can ensure accuracy of an initial position of the calibration device, laying a reliable foundation for subsequent fine calibration.

In some embodiments, the calibration device 100 is provided with a recessed groove 3 and a pair of light-transmitting holes 4. The center point P of the calibration device 100 is accommodated in the recessed groove 3. One end of each of the pair of light-transmitting holes 4 penetrates the calibration device 100, and another end of each of the pair of light-transmitting holes 4 communicates with the recessed groove 3. The pair of light-transmitting holes 4 and the recessed groove 3 form the positioning channel 5 together. The pair of positioning surfaces 61 block the pair of light-transmitting holes 4. The positioning channel 5 intersects with one of the at least two optical path channels 1; and/or, a shape of the positioning portion 611 is a crosshair structure (e.g., a crosshair), and an intersection point of the crosshair is the central positioning point N.

It should be noted that the count of the light-transmitting holes 4 is merely an example. The count of the light-transmitting holes 4 may be adaptively set according to requirements. For example, two pairs, three pairs, or more pairs of light-transmitting holes may be provided, which respectively communicate with the recessed groove or penetrate through the calibration device 100 to form different optical path channels.

More descriptions regarding the central positioning point N, the recessed groove 3, and the light-transmitting holes 4 may be found in the related description above.

In some embodiments, the positioning channel is constructed using the recessed groove and the light-transmitting holes. This structure is simple and can ensure precise intersection of the positioning channel and the optical path channel at the center point P. The positioning portion 611 with the crosshair structure provides a high-precision visual alignment reference, achieving fast and accurate initial positioning.

In some embodiments, as shown in FIG. 1, an image-guided system includes the calibration device 100, at least one pair of ray generating devices 200, configured to generate the rays passing through the at least two optical path channels of the calibration device 100. The rays generated by the at least one pair of ray generating devices 200 intersect each other, rays passing through the at least two optical path channels of the calibration device 100 and generated by the at least one pair of ray generating devices intersect with each other; at least one pair of image processing devices 300, configured to receive the rays passing through the at least two optical path channels of the calibration device and generate image information. More descriptions regarding the calibration device 100 may be found in the related description above.

The ray generating device 200 is a device configured to generate rays capable of passing through the optical path channel 1 of the calibration device 100. For example, the ray generating device 200 is an X-ray tube, an electron gun in a linear accelerator, or other components capable of generating diagnostic-level X-rays, or the like.

The ray generating device 200 corresponds one-to-one with the optical path channel 1.

Specifically, the ray generating device 200 is configured as at least one pair. The at least one pair of ray generating devices 200 correspond in position to at least one pair of optical path channels 1 in the calibration device 100. In some embodiments, the rays generated by the at least one pair of ray generating devices 200 intersect each other to pass through at least one pair of intersecting optical path channels 1 in the calibration device 100. It should be noted that the count of the ray generating devices 200 is merely an example. The count of the ray generating devices 200 may be adaptively set according to requirements. For example, the count of the ray generating devices 200 is one pair, two pairs, or more pairs, etc.

The image processing device 300 is a device configured to receive the rays passing through the optical path channel 1 of the calibration device 100 and generate image information. For example, the image processing device 300 includes a flat panel detector, an image intensifier, or other imaging equipment capable of converting an X-ray intensity distribution into a digital signal, etc.

Specifically, the image processing device 300 is configured as at least one pair. The at least one pair of image processing devices 300 correspond in position to at least one pair of optical path channels 1 in the calibration device 100. It should be noted that the count of the image processing devices 300 is merely an example. The count of the image processing devices 300 may be adaptively set according to requirements. For example, the count of the image processing devices 300 is one pair, two pairs, or more pairs, etc.

The image information refers to digital image data generated by the image processing device 300 (e.g., the flat panel detector) after receiving the rays passing through the calibration device 100.

In some embodiments, the image information includes projection images of the two imaging surfaces 21, the projection images include the marking portions 211 (including the central marking point M) of the two imaging surfaces 21. For example, the image information is an X-ray digital image containing crosshair mark projections of two sets of imaging surfaces 21, etc.

In some embodiments, because the two imaging surfaces 21 are disposed at opposite ends of the same optical path channel 1 among the at least two optical path channels 1, when a ray passes through the optical path channel 1, the ray also passes through the two imaging surfaces 21 disposed at the opposite ends of the optical path channel 1, forming projection images of the two imaging surfaces 21. Simultaneously, the marking portions 211 of the imaging surfaces 21 are also projected, i.e., the projection images include the marking portions 211 of the two imaging surfaces 21.

In some embodiments, through the calibration device, the ray generating devices, and the image processing devices, utilizing intersecting optical path channels and imaging surfaces, projection data of a pair of ray sources can be simultaneously acquired in a single imaging operation, which enables efficient and precise calculation of positional offset information of each ray generating device and each image processing device, achieving rapid calibration of overall geometric accuracy of the image-guided system.

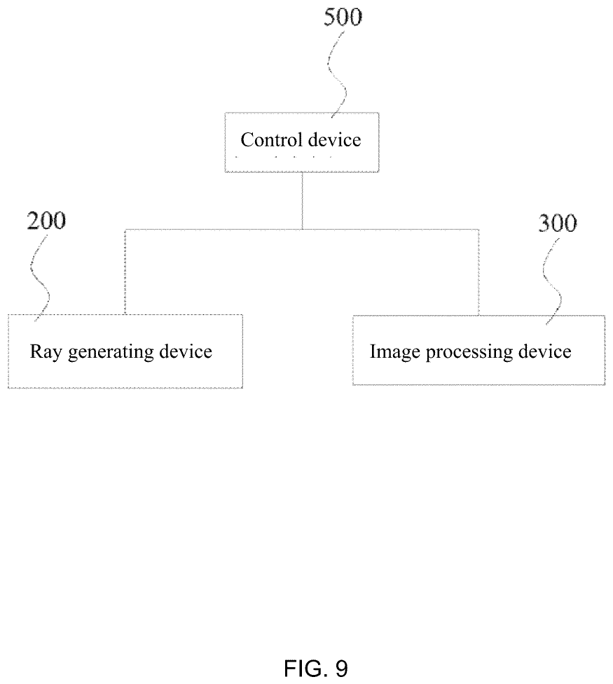

FIG. 9 is a schematic diagram of a connection of a control device, a ray generating device, and an image processing device according to some embodiments of the present disclosure.

In some embodiments, as shown in FIG. 9, the image-guided system further includes a control device 500, the control device 500 is configured to control movement of at least one of the at least one pair of ray generating devices 200 or the at least one pair of image processing devices 300; and/or, the at least one pair of ray generating devices 200 are X-ray tubes, and the at least one pair of image processing devices 300 are flat panel detectors.

The control device 500 is a device configured to control movement of the ray generating device 200 and/or the image processing device 300. For example, the control device 500 controls movement and rotation of the ray generating device 200 and/or the image processing device 300 along X, Y, and Z axes. The X axis is a horizontal line, and the horizontal line passes through the pair of ray generating devices 200 or the pair of image processing devices 300 along an extension path. The Y axis is another horizontal line perpendicular to the X axis. The Z axis is a vertical line.

In some embodiments, the control device 500 employs an automated device (e.g., a servo motor, an electric push rod, or the like) to automatically control movement of the ray generating device 200 and/or the image processing device 300 based on a processing result of the data processing device. The data processing device refers to a computing device in the image-guided system configured to receive, analyze, and calculate image information and generate positional offset information. For example, the data processing device may be a dedicated image processing computer, an industrial PC integrated in a control cabinet, or an embedded processing device based on a central processing unit (CPU)/graphics processing unit (GPU), or the like. More descriptions regarding the data processing device may be found in the following sections and related descriptions.

In some embodiments, the control device 500 is a manually adjustable mechanical mechanism (e.g., a graduated handwheel, or the like). When an offset of the ray generating device 200 and/or the image processing device 300 is detected, the control device 500 is manually adjusted to adjust the position of the ray generating device 200 and/or the image processing device 300.

In some embodiments, the ray generating device 200 is an X-ray tube, or the like. The X-ray tube is configured to generate X-rays.

In some embodiments, the image processing device 300 is a flat panel detector, or the like. The flat panel detector is configured to receive rays, convert optical signals into electrical signals containing image information, and transmit the electrical signals to the data processing device for data processing.

In some embodiments, by incorporating the control device, automated or semi-automated correction of positional offsets of a ray source and a detector is achieved, significantly improving calibration efficiency and accuracy. Using a combination of the X-ray tube and the flat panel detector ensures imaging quality of the system and reliability of the calibration method.

Step S1: The calibration device 100 is installed to the image-guided system, and a center point P of the calibration device 100 is aligned with a target point of the image-guided system. The target point of the image-guided system is a position of a lesion of a patient during radiotherapy. The target point of the image-guided system is input by a user. The user is an individual using the image-guided system, or the like.

Step S2: Rays emitted by at least one pair of ray generating devices 200, wherein the rays pass through the calibration device 100 and are received by the at least one pair of image processing devices 300, and the at least one pair of image processing devices 300 generate the image information of the received rays. More descriptions regarding the image information may be found in the above sections and related descriptions.

Step S3: The image information is transmitted to a data processing device by the at least one pair of image processing devices 300. Positional offset information of at least one of the at least one pair of ray generating devices 200 or the at least one pair of image processing devices 300 is obtained by the data processing device based on the image information.

The positional offset information is data used to quantify a deviation between an actual position and an ideal position of the ray generating device 200 and/or the image processing device 300. The ideal position refers to a spatial position where the ray generating device 200 and/or the image processing device 300 should be located according to a theoretical design of the image-guided system to ensure geometric accuracy of the image-guided system.

In some embodiments, the positional offset information includes a translational offset (e.g., distance values along X and Y directions) and/or a rotational offset (e.g., a rotation angle about a Z axis), or the like. For example, the positional offset information includes command data such as the ray generating device 200 needing to move positively by 2.1 mm along the X axis, or the image processing device 300 needing to rotate counterclockwise by 0.5°, or the like.

In some embodiments, the image information generated by the image processing device 300 after receiving the rays includes projection images of the two imaging surfaces 21. If two central marking points M coincide in the projection images of the two imaging surfaces 21, the rays pass through the center point P of the calibration device 100, and a position of the ray generating device 200 emitting the rays is accurate, i.e., the translational offset and the rotational offset in the positional offset information are zero. If the two central marking points M do not coincide in the projections of the two imaging surfaces 21, the rays do not pass through the center point P of the calibration device 100, and the ray generating device 200 emitting the rays has an offset. The positional offset information is calculated based on a geometric positional relationship between the two non-coincident central marking points M and a geometric center of the projection images. More descriptions regarding how to obtain the positional offset information may be found in the following related content.

Step S4: A position of the at least one pair of ray generating devices 200 or the at least one pair of image processing devices 300 is adjusted based on the positional offset information.

In some embodiments, if the positional offset information includes a translational offset, the position of the ray generating device 200 is adjusted. For example, if the positional offset information includes a rotational offset, the position of the image processing device 300 is adjusted along a direction opposite to the rotational offset (e.g., adjusted counterclockwise if the rotational offset is 0.3 degrees clockwise) according to a preset rotation adjustment amount (e.g., 0.1 degree, 0.5 degree, or 1 degree, or the like).

Step S5: Step S3 to step S4 are repeated until the position of at least one of the at least one pair of ray generating device 200 or the at least one pair of image processing device 300 is adjusted to a designated position.

The designated position refers to a position that the ray generating device 200 and the image processing device 300 need to reach after calibration to meet geometric accuracy requirements of the image-guided system. For example, the designated position is a position where the translational offset in the positional offset information is zero and the rotational offset is zero.

In some embodiments, when the position of the at least one pair of ray generating devices 200 and/or the at least one pair of image processing devices 300 after adjustment in step S4 is not at the designated position, step S3 to step S4 are repeated until the ray generating device 200 and/or the image processing device 300 is adjusted to the designated position, and adjustment is stopped.

In some embodiments, through the position calibration method, positional offset of the ray generating device and perpendicularity offset between the rays and the image processing device can be calibrated, improving positional accuracy of the ray generating device and the image processing device, thereby improving surgical accuracy for a lesion of a patient during surgery.

In some embodiments, step S2 includes the at least one pair of ray generating devices 200 emitting rays, the rays passing through the two imaging surfaces 21 of the calibration device 100 and being received by the at least one pair of image processing devices 300.

In some embodiments, the rays pass through the two imaging surfaces 21 of the calibration device 100, the at least one pair of image processing devices 300 corresponds one-to-one with the at least one pair of ray generating devices, each image processing device 300 receives rays generated by one of the at least one pair of ray generating device corresponding to each image processing device 300 and generates image information including two imaging surfaces projections.

In some embodiments, one image processing device 300 corresponds to one ray generating device 200. That is, one image processing device 300 is configured to receive rays emitted by one ray generating device 200, and the image processing device 300 and the ray generating device 200 are located on opposite sides of one optical path channel 1. Rays emitted by one ray generating device 200 are received by a corresponding one image processing device 300, and the image processing device 300 can form image information. Because the rays emitted by the ray generating device 200 are partially blocked when passing through the imaging surface 21 of the calibration device 100 due to absorption or obstruction of the rays by a marking portion 211 (e.g., a crosshair structure) on the imaging surface 21, the image information formed by the image processing device 300 includes projections of the two imaging surfaces 21 through which the rays pass.

The imaging surface projection refers to an image of the marking portion formed on the image processing device (e.g., the flat panel detector) when rays pass through the imaging surface of the calibration device, due to obstruction or absorption of the rays by the marking portion on the imaging surface. For example, the imaging surface projection may be a cross-shaped image with distinct grayscale contrast formed by a crosshair marking portion on the detector, or a circular light spot image formed by a point-shaped marking, or the like.

The image information of the imaging surface projection refers to digitized data generated by the image processing device that contains the aforementioned projection. For example, the image information may be an X-ray digital image file containing two sets of cross-mark projections of the imaging surfaces, or an array of pixel coordinate data of center points of each projection extracted therefrom, or the like.

In some embodiments, by having one pair of ray generating devices and one pair of image processing devices work in one-to-one correspondence, and ensuring each ray beam penetrates the two imaging surfaces of the same optical path channel on the calibration device, a single imaging operation can simultaneously capture two marking projections with a fixed spatial relationship in each image, providing a necessary and sufficient image data foundation for accurately calculating a translational positional offset of the ray source, greatly improving calibration efficiency.

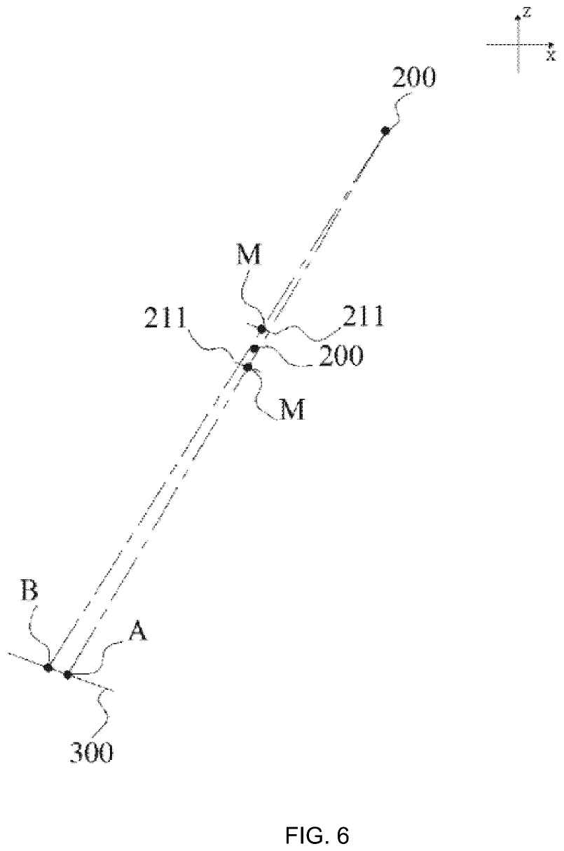

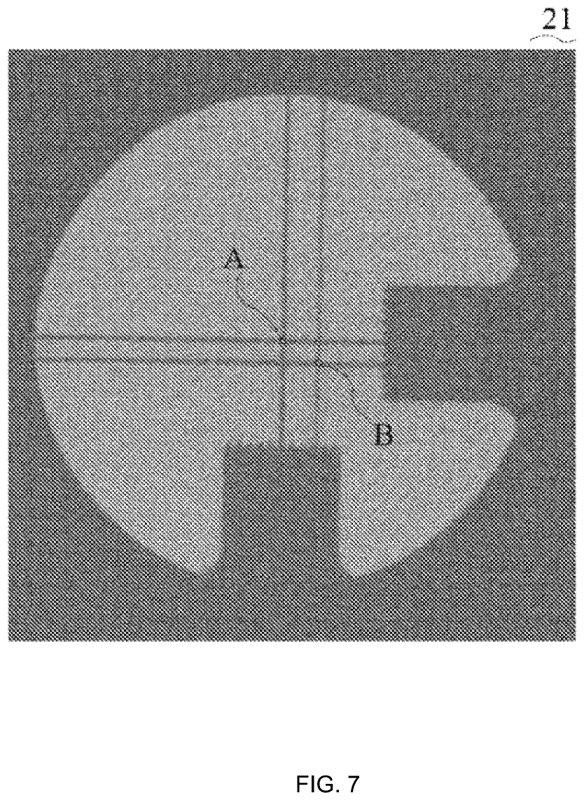

FIG. 6 is a schematic diagram of partial ray irradiation paths when a ray generating device is offset according to some embodiments of the present disclosure. FIG. 7 is a schematic diagram of image information obtained by partial image processing devices when a ray generating device is offset according to some embodiments of the present disclosure.

In the step S3, the data processing device determining the positional offset information of the at least one pair of ray generating devices and/or the at least one pair of image processing devices based on the image information includes:

In some embodiments, designating a center point of an image generated by the at least one pair of image processing devices as a first origin point, a coordinate point A of a central marking point of one of the two imaging surface projection in the image information that is closer to the target point of the image-guided system is obtained as (X1, Y1), and a coordinate point B of a central marking point of one of the two imaging surface projection in the image information that is far from the target point of the image-guided system is obtained as (X2, Y2), wherein a positional offset of the at least one pair of ray generating devices in an X axis direction is (X1+X2)/2, and a positional offset of the at least one pair of ray generating devices in a Y axis direction is (Y1+Y2)/2.

In some embodiments, in response to X1>X2, the at least one pair of ray generating devices are adjusted to move a distance of (X1+X2)/2 along a negative direction of an X axis. In response to X1<X2, the at least one pair of ray generating devices 200 are adjusted to move a distance of (X1+X2)/2 along a positive direction of the X axis. In response to Y1>Y2, the at least one pair of ray generating devices are adjusted to move a distance of (Y1+Y2)/2 along a negative direction of an Y axis. In response to Y1<Y2, the at least one pair of ray generating devices are adjusted to move a distance of (Y1+Y2)/2 along a positive direction of the Y axis.

The central marking point of the imaging surface projection closer to the target point of the image-guided system refers to the imaging surface of the two imaging surfaces that is closer to the target point of the image-guided system (e.g., the position of the patient's lesion). The central marking point of the imaging surface projection farther from the target point of the image-guided system refers to the imaging surface of the two imaging surfaces that is farther from the target point of the image-guided system (e.g., the position of the patient's lesion). As shown in FIG. 6, a point closer to the image processing device 300 is coordinate point A (X1, Y1) because the point is closer to the target point of the image-guided system in spatial position. A point farther from the image processing device 300 is coordinate point B (X2, Y2) because the point is farther from the target point of the image-guided system in spatial position.

In some embodiments, as shown in FIG. 6 and FIG. 7, using the center point of the image generated by the image processing device as the first origin point, using mutually perpendicular horizontal lines as the X axis and the Y axis, and using a vertical line as a Z axis, the X axis is a straight-line direction passing through the pair of image processing devices, a coordinate point A of a central marking point M of one imaging surface projection of two imaging surface projections in the image information that is closer to the target point of the image-guided system is obtained as (X1, Y1), and a coordinate point B of a central marking point M of one imaging surface projection of the two imaging surface projections in the image information that is farther from the target point of the image-guided system is obtained as (X2, Y2). When the position of the ray generating device is accurate, rays emitted by the ray generating device 200 pass through marking points on the pair of imaging surfaces 21 and the center point P of the calibration device. At this time, coordinate point A and coordinate point B coincide (i.e., X1=X2, Y1=Y2). If coordinate point A and coordinate point B do not coincide, the ray generating device is determined to have an offset. A positional offset of the ray generating device in the X axis direction is calculated as (X1+X2)/2, and a positional offset of the ray generating device in the Y axis direction is calculated as (Y1+Y2)/2.

In step S4 described above, the adjusting the position of the at least one pair of ray generating devices and/or the at least one pair of image processing devices according to the positional offset information includes:

In some embodiments, if X1>X2, the ray generating device is offset to the left (i.e., the positive direction of the X axis), causing a proximal marking point A to be offset to the right (i.e., the negative direction of the X axis) relative to a distal marking point B. In this case, the ray generating device is adjusted to move a distance of (X1+X2)/2 along the negative direction of the X axis. If X1<X2, the ray generating device is offset to the right (i.e., the negative direction of the X axis), causing the proximal marking point A to be offset to the left (i.e., the positive direction of the X axis) relative to the distal marking point B. In this case, the ray generating device is adjusted to move a distance of (X1+X2)/2 along the positive direction of the X axis. If Y1>Y2, the ray generating device is offset downward (i.e., the positive direction of the Y axis), causing the proximal marking point A to be offset upward (i.e., the negative direction of the Y axis) relative to the distal marking point B. In this case, the ray generating device is adjusted to move a distance of (Y1+Y2)/2 along the negative direction of the Y axis. If Y1<Y2, the ray generating device is offset upward (i.e., the negative direction of the Y axis), causing the proximal marking point A to be offset downward (i.e., the positive direction of the Y axis) relative to the distal marking point B. In this case, the ray generating device is adjusted to move a distance of (Y1+Y2)/2 along the positive direction of the Y axis.

In some embodiments, by precisely quantifying projection coordinate deviations of two marking points and clarifying a relationship between an offset direction and an adjustment direction, quantitative, automated closed-loop calibration for a position offset of the ray generating device is achieved. Thus, complex spatial geometric errors are converted into simple image coordinate calculations, significantly improving calibration accuracy, efficiency, and repeatability.

In the step S5, until the position of at least one of the at least one pair of ray generating devices or the at least one pair of image processing devices is adjusted to a designated position includes:

In some embodiments, steps S3 to S4 are repeated until the positional offset of the at least one pair of ray generating devices in the X axis direction and the positional offset of the at least one pair of ray generating devices in the Y axis direction are both less than a specified threshold. Then, the at least one pair of ray generating devices 200 are adjusted to a designated position.

The specified threshold is preset based on manual experience. Merely by way of example, the specified threshold is 1 mm, etc.

More descriptions regarding the designated position may be found in the related description above.

In some embodiments, by introducing the specified threshold as an objective quantitative standard for terminating calibration, calibration accuracy is ensured to be controllable and consistent, avoiding over-adjustment or insufficient calibration. Thus, reliability and repeatability of calibration results are significantly improved.

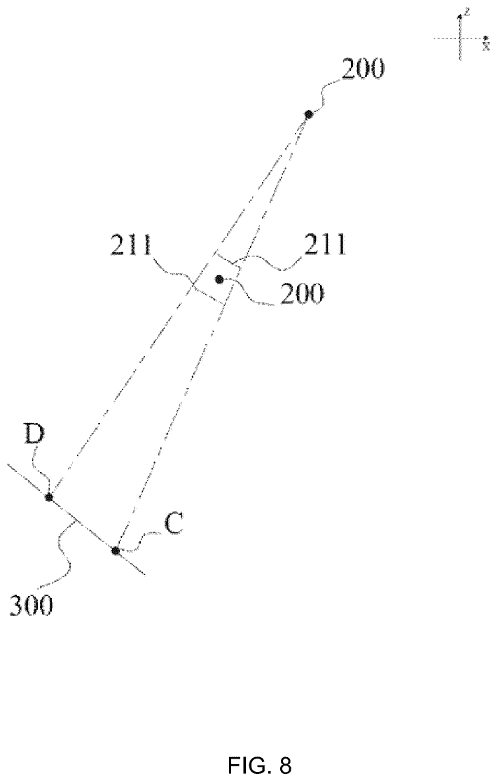

FIG. 8 is a schematic diagram of partial ray irradiation paths when an image processing device is offset according to some embodiments of the present disclosure.

In the step S3, the transmitting, by the at least one pair of image processing devices, the image information to a data processing device, and obtaining, by the data processing device, positional offset information of at least one of the at least one pair of ray generating devices or the at least one pair of image processing devices based on the image information includes:

In some embodiments, as shown in FIG. 8, a central marking point of one of the imaging surface projections in an image generated by the at least one pair of image processing devices is designated as a second origin point. A coordinate of a first endpoint C of the target point in the image-guided system (e.g., a lesion position), the first endpoint C is adjacent to the marking portion within the one of the imaging surface projection, is obtained as (X3, Y3). A coordinate of a second endpoint D of the target point in the image-guided system, the first endpoint C is far from the marking portion within the one of the imaging surface projection, is obtained as (X4, Y4). A ratio of a distance between a first marking point in the marking portion that is formed by a projection of the first endpoint C and the central marking point to a distance between a second marking point in the marking portion that is formed by a projection of the second endpoint D and the central marking point is a:b. In response to X3>X4, and the ratio of the distance between the first endpoint C and the second origin point to the distance between the second endpoint D and the second origin point being less than a:b, determining that the image processing device including the second origin point is offset counterclockwise. In response to X3>X4 and the ratio of the distance between the first endpoint C and the second origin point to the distance between the second endpoint D and the second origin point being less than a:b, the image processing device containing the second origin point is determined to be offset counterclockwise. In response to X3<X4, and the ratio of the distance between the first endpoint C and the second origin point to the distance between the second endpoint D and the second origin point being greater than a:b, determining that the image processing device including the second origin point is offset counterclockwise; in response to X3<X4, and the ratio of the distance between the first endpoint C and the second origin point to the distance between the second endpoint D and the second origin point being less than a:b, determining that the image processing device including the second origin point is offset clockwise.

In some embodiments, in response to the image processing device including the second origin point being offset counterclockwise, the image processing device including the second origin point clockwise is rotated by the rated angle about the rotation axis; wherein the rotation axis is a straight line perpendicular to a connection line between the first endpoint C and the second endpoint D and passing through the second origin point.

In some embodiments, the marking portion includes a plurality of endpoints. As another example, the marking portion is a crosshair structure, and four endpoints of the crosshair structure are the four endpoints of the marking portion.

In some embodiments, an endpoint forming the first marking point and an endpoint forming the second marking point are located in a same marking portion. The marking portion and the central marking point corresponding to the second origin point are located on a same imaging surface.

In some embodiments, as shown in FIG. 8, the central marking point of an imaging surface projection in an image generated by the at least one pair of image processing devices is used as the second origin point. Mutually perpendicular horizontal lines are used as the X axis and the Y axis, and a vertical line is used as a Z axis. The X axis is specifically a straight line passing through the pair of image processing devices. A coordinate of the first endpoint C in the marking portion of the imaging surface projection where the second origin point is located is obtained as (X3, Y3). A coordinate of the second endpoint D in the marking portion of the imaging surface projection where the second origin point is located is obtained as (X4, Y4).

The first endpoint C refers to an image of an endpoint of the marking portion (e.g., a crosshair) on the imaging surface in a projection image, which is close to the target point of the image-guided system (i.e., the isocenter).

The second endpoint D refers to an image of an endpoint of the marking portion (e.g., a crosshair, etc.) on the imaging surface in the projection image, which is away from the target point of the image-guided system (i.e., the isocenter).

For example, assuming the marking portion is a crosshair structure, a direction horizontally to the right is the positive direction of the x-axis, and a direction vertically upward perpendicular to the x-axis is the positive direction of the y-axis. As shown in FIG. 8, X-rays are emitted from the ray generating device 200, sequentially pass through a first imaging surface, the target point of the image-guided system (i.e., the isocenter), and a second imaging surface, forming a projection containing two crosshair structures. Assuming the central marking point of the second imaging surface projection is used as the second origin point, and the image processing device has a counterclockwise rotation offset. Due to the rotation offset of the image processing device, the image processing device is not perpendicular to the ray beam, causing distances from four endpoints of the crosshair structure on the second imaging surface received by the image processing device to the target point of the image-guided system to be different. Deformation along the x-axis is much greater than along the y-axis. The positive direction of the x-axis is severely compressed, and the negative direction of the x-axis is severely stretched. In this case, the first endpoint C is the endpoint located in the positive direction of the x-axis. The second endpoint D is the endpoint located in the negative direction of the x-axis.

A ratio of a distance between the first marking point and the central marking point M to a distance between the second marking point and the central marking point M is a:b. The first marking point refers to an endpoint of the marking portion that projects to form the first endpoint C. The second marking point refers to an endpoint of the marking portion that projects to form the second endpoint D.

In some embodiments, when a receiving plane of the image processing device 300 is perpendicular to rays emitted by a corresponding ray generating device 200, an actual physical distance between the first marking point and the central marking point M on the imaging surface 21 is converted into a pixel distance between the first endpoint C and the second origin point in the projection image according to a certain proportion. An actual physical distance between the second marking point and the central marking point M on the imaging surface 21 is converted into a pixel distance between the second endpoint D and the second origin point in the projection image according to a certain proportion. Because the actual physical distance between the first marking point and the central marking point on the imaging surface and the actual physical distance between the second marking point and the central marking point on the imaging surface are magnified by the same proportion in the projection image. That is, a ratio of the pixel distance between the first endpoint C and the second origin point to the pixel distance between the second endpoint D and the second origin point should also be equal to a:b. If the ratio of the pixel distance between the first endpoint C and the second origin point to the pixel distance between the second endpoint D and the second origin point is not equal to a:b, the image processing device 300 is determined to have an offset.

In some embodiments, if X3>X4 and a ratio of a distance between the first endpoint C and the second origin point to a distance between the second endpoint D and the second origin point is greater than a:b, the image processing device containing the second origin point is determined to be offset clockwise. Here, X3>X4 indicates that the projection of the first endpoint C is located to the right of the projection of the second endpoint D. The ratio of the distance between the first endpoint C and the second origin point to the distance between the second endpoint D and the second origin point being greater than a:b indicates that an end of the central marking point farther from the ray source is compressed in the image. In this case, the image processing device containing the second origin point is determined to have a clockwise offset.

In some embodiments, if X3>X4 and a ratio of a distance between the first endpoint C and the second origin point to a distance between the second endpoint D and the second origin point is less than a:b, the image processing device containing the second origin point is determined to be offset counterclockwise. Here, X3>X4 indicates that the projection of the first endpoint C is located to the right of the projection of the second endpoint D. The ratio of the distance between the first endpoint C and the second origin point to the distance between the second endpoint D and the second origin point being less than a:b indicates that an end of the central marking point farther from the ray source is stretched in the image. In this case, the image processing device containing the second origin point is determined to have a counterclockwise offset.

In some embodiments, if X3<X4 and a ratio of a distance between the first endpoint C and the second origin point to a distance between the second endpoint D and the second origin point is greater than a:b, the image processing device containing the second origin point is determined to be offset counterclockwise. Here, X3<X4 indicates that the projection of the first endpoint C is located to the left of the projection of the second endpoint D. The ratio of the distance between the first endpoint C and the second origin point to the distance between the second endpoint D and the second origin point being greater than a:b indicates that an end of the central marking point farther from the ray source is compressed in the image. In this case, the image processing device containing the second origin point is determined to have a counterclockwise offset.

In some embodiments, if X3<X4 and a ratio of a distance between the first endpoint C and the second origin point to a distance between the second endpoint D and the second origin point is less than a:b, the image processing device containing the second origin point is determined to be offset clockwise. Here, X3<X4 indicates that the projection of the first endpoint C is located to the left of the projection of the second endpoint D. The ratio of the distance between the first endpoint C and the second origin point to the distance between the second endpoint D and the second origin point being less than a:b indicates that an end of the central marking point farther from the ray source is stretched in the image. In this case, the image processing device containing the second origin point is determined to have a clockwise offset.

In some embodiments, the marking portion 211 is selected as a regular crosshair structure, i.e., distances between each endpoint of the marking portion 211 and the central marking point M are the same, so that a:b is 1:1. Thus, during determination: In some embodiments, if X3>X4 and a distance between the first endpoint C and the second origin point is greater than a distance between the second endpoint D and the second origin point, the image processing device containing the second origin point is determined to be offset clockwise. In some embodiments, if X3>X4 and the distance between the first endpoint C and the second origin point is less than the distance between the second endpoint D and the second origin point, the image processing device containing the second origin point is determined to be offset counterclockwise. In some embodiments, if X3<X4 and the distance between the first endpoint C and the second origin point is greater than the distance between the second endpoint D and the second origin point, the image processing device 300 containing the second origin point is determined to be offset counterclockwise. In some embodiments, if X3<X4 and the distance between the first endpoint C and the second origin point is less than the distance between the second endpoint D and the second origin point, the image processing device 300 containing the second origin point is determined to be offset clockwise.

In some embodiments, the step S4 specifically includes that:

If the image processing device 300 is offset clockwise, the image processing device 300 is rotated counterclockwise by a rated angle about a rotation axis. If the image processing device 300 is offset counterclockwise, the image processing device 300 is rotated clockwise by the rated angle about the rotation axis. The rotation axis is a straight line perpendicular to a connection line between the first endpoint C and the second endpoint D and passing through the second origin point. The rated angle is a preset angle based on manual experience. Merely by way of example, the rated angle is an angle within a range of 0.05° to 1°, etc.

In some embodiments, a rotational offset of an image processing device (e.g., a detector) that is difficult to measure directly is converted into a precisely calculable logical judgment. Furthermore, a correction instruction in an opposite direction is provided (clockwise offset leads to counterclockwise adjustment), achieving efficient and precise closed-loop calibration for perpendicularity between a detector plane and a ray beam.

In some embodiments, the step S5 specifically includes that: steps S3 to S4 is repeated after the image processing device 300 including the second origin point is rotated by the rated angle, an offset direction of the image processing device 300 including the second origin point changing (e.g., from clockwise offset to counterclockwise offset). Then, the image processing device 300 including the second origin point is adjusted to the designated position.

In some embodiments, when the detector is rotated by the rated angle, if the offset direction of the detector changes, it proves that calibration has crossed a theoretical zero-offset point. At this point, an actual position of the detector is very close to an ideal perpendicular state. Through this calibration method, complex absolute angle measurement is converted into simple direction change judgment, reducing dependence on control precision and simplifying algorithms. The calibration process becomes fast, stable, and easy to achieve automated closed-loop control, effectively avoiding repeated oscillation near a zero position or insufficient calibration.

In some embodiments, the calibration device 100 further includes: a positioning channel 5 penetrating through the calibration device 100, wherein the positioning channel 5 is configured to allow light to pass through. The positioning channel 5 extends along a center line of the calibration device 100, and the positioning channel 5 passes through a center point of the calibration device 100. A positioning assembly 6 includes a pair of positioning surfaces 61, wherein the pair of positioning surfaces 61 is disposed at opposite ends of the positioning channel 5. The pair of positioning surfaces 61 are provided with positioning portions 611. The positioning portions 611 are provided with central positioning points N, and a connection line of the pair of central positioning points N of the pair of positioning surfaces 61 passes through the center point of the calibration device 100. The installing the calibration device 100 to the image-guided system and aligning the center point of the calibration device 100 with a target point of the image-guided system includes: using a survey tool to form a horizontal optical path passing through the target point of the image-guided system, the calibration device 100 is installed to the image-guided system, and the pair of central positioning points N of the calibration device 100 coincide with each other along a projection of the horizontal optical path of the survey tool are aligned. The calibration device 100, so that rays generated by the at least one pair of ray generating devices 200 pass through at least two imaging surfaces 21 of the calibration device 100. More descriptions regarding the positioning channel 5 and the positioning assembly 6 may be found in the related description above.

In some embodiments, the step S1 specifically includes: fixing the calibration device 100 to the image-guided system through two mounting holes 7 of the calibration device 100, moving the calibration sphere 9 from the calibration hole 8 of the calibration device 100 into the recessed groove 3 of the calibration device 100, and using a survey tool (e.g., a laser collimator, a theodolite, or the like) to form a horizontal optical path passing through the target point of the image-guided system, i.e., passing through the calibration sphere 9. For example, a theodolite is used to observe the calibration sphere 9 in a horizontal direction, a line parallel to an observation line of the theodolite passes through the at least one pair of ray generating devices 200 or the at least one pair of image processing devices 300, forming the target point. Subsequently, adjusting positions of the central positioning points N of the pair of positioning portions 611 in the calibration device 100 until the pair of central positioning points N and the calibration sphere 9 coincide under an observation perspective of the theodolite, enabling the center point P of the calibration device 100 to coincide with the calibration sphere 9, at this time the center point P of the calibration device 100 coincides with the isocenter of the image-guided system, marking completion of positioning calibration of the calibration device 100 in position. Subsequently, turning on the ray generating device 200, rotating the calibration device 100 to cause a ray generated by the ray generating device 200 to pass through at least one pair of imaging surfaces 21 located on one optical path channel 1 in the calibration device 100, thereby completing installation and positioning of the calibration device 100. When rotating the calibration device 100, the calibration device 100 rotates about an axis passing through the pair of central positioning points N.

In some embodiments, a visual optical reference is formed through the positioning channel 5 and the positioning assembly 6, and with the assistance of a laser beam from an external survey tool, rapid and precise initial alignment between the calibration device and the target point of the system is achieved, effectively avoiding calibration errors caused by initial installation deviations, and significantly improving efficiency and reliability of the overall calibration process.

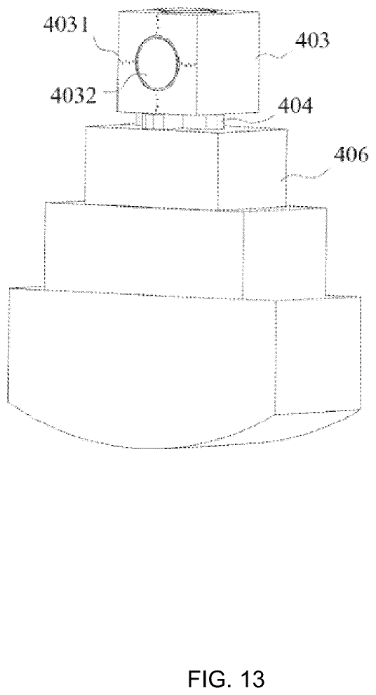

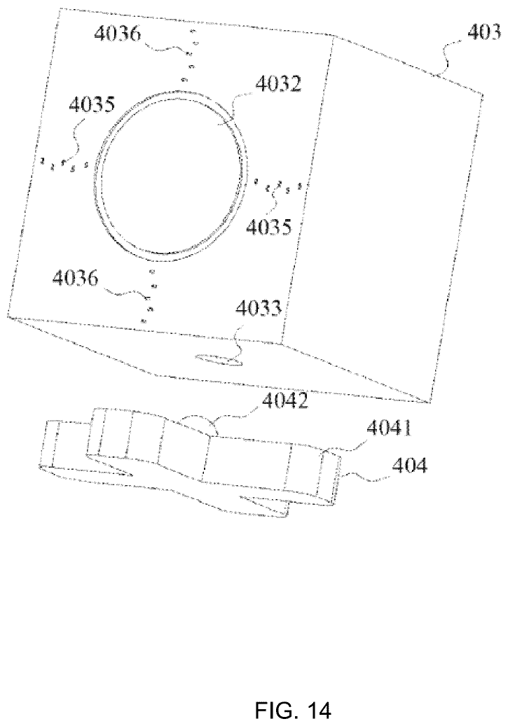

FIG. 13 is a schematic diagram of a structure when a treatment head, a mechanical isocenter jig, and a laser beam verification phantom are connected according to some embodiments of the present disclosure. FIG. 14 is an exploded schematic diagram of a mechanical isocenter jig and a laser beam verification phantom according to some embodiments of the present disclosure.

To ensure installation accuracy of the laser lamp 402, and to ensure that laser beams generated by a plurality of laser lamps 402 intersect at the isocenter position. Some embodiments of the present disclosure provide a treatment room positioning system, which is formed in a treatment room, a treatment positioning system is installed in the treatment room, and the treatment positioning system is configured to perform radiation therapy on a patient tumor located at an isocenter position of the treatment positioning system. The treatment positioning system (hereinafter also referred to as the treatment system) adopts an existing structure, e.g., a proton therapy system or the like.

Some embodiments of the present disclosure provide a calibration method for a treatment room positioning system, the treatment room positioning system is configured to assist in positioning of a target region in a treatment system. The calibration method includes the following steps S01-S05.

Step S01: A plurality of laser lamps 402 are installed in the treatment room.

The treatment room is a dedicated shielded space for performing radiation therapy. Merely by way of example, a radiation therapy room equipped with a linear accelerator or the like.

The count of the plurality of laser lamps is preset according to actual requirements, e.g., three or the like.

In some embodiments, a position measuring instrument 405 is first used to determine a pair of mounting positions having the same height and being horizontally symmetrical in the treatment room positioning system. The position measuring instrument 405 employs a laser tracker. Each mounting position is configured to install one laser lamp 402. Merely by way of example, a pair of mounting positions having the same height and being left-right symmetrical in the treatment room, the pair of mounting positions are correspondingly disposed on left and right side wall bodies of the treatment room. By positioning the pair of horizontally symmetrical mounting positions, a pair of laser lamps 402 installed at the pair of mounting positions are installed horizontally symmetrically, facilitating adjustment of positions of the laser lamps 402.

In some embodiments, the laser lamp 402 is 1.23-1.29 m from a floor of the treatment room or the like. When determining the mounting position, based on a position of the isocenter position of the treatment positioning system, a projection of the position on the wall body is obtained, and a location where the projection is located is used as the mounting position. The position of the isocenter position of the treatment positioning system or the like is determined based on a position of the treatment head 406, e.g., the position of the isocenter position of the treatment positioning system or the like is a position where the treatment head 406 is rotated to 180°, i.e., a position where the treatment head 406 is rotated to the floor of the treatment room, and a height direction of the treatment head 406 is perpendicular to the floor of the treatment room. The position of the isocenter position of the treatment positioning system is higher than the position of the treatment head 406.

In some embodiments, a mounting position is disposed above the treatment room, and one laser lamp 402 is installed at the mounting position; the mounting position located above the treatment room is disposed on a ceiling of the treatment room.

In some embodiments, it should be noted that when a quantity of marks preset based on a position of a patient tumor is more than three, e.g., four or five, a quantity of the laser lamps 402 is correspondingly set to four or five. When the quantity of the laser lamps 402 is four, three of the laser lamps 402 are disposed on left and right side wall bodies and the ceiling of the treatment room, a mounting position is disposed on a front side wall body or a rear side wall body of the treatment room, and another laser lamp 402 is installed at the mounting position on the front side wall body or the rear side wall body; when the quantity of the laser lamps 402 is five, three of the laser lamps 402 are disposed on the left and right side wall bodies and the ceiling of the treatment room, mounting positions are respectively disposed on the front side wall body and the rear side wall body of the treatment room, and the remaining two laser lamps 402 are respectively installed at the mounting positions on the front side wall body and the rear side wall body. A pair of mounting positions formed by the front side wall body and the rear side wall body of the treatment room are a pair of mounting positions having the same height and being front-rear symmetrical in the treatment room, and the pair of mounting positions having the same height and being front-rear symmetrical have the same height as the mounting positions on the left and right side wall bodies of the treatment room.

In some embodiments, after the laser lamp 402 is installed on the wall body 401, an electronic display level is used to measure horizontality and verticality of the laser lamp 402 relative to a wall surface or a bottom surface of the treatment room, and a tolerance of the horizontality and the verticality of the laser lamp 402 is maintained within 0.01 mm. More descriptions about the laser lamp may be found in related descriptions below.

Step S02: the mechanical isocenter jig 404 is installed on the treatment head 406, wherein the mechanical isocenter jig 404 is preset with a central reference object, and the central reference object 4042 is configured as a spatial physical representation of the isocenter position of the treatment system to configure the treatment system.

In some embodiments, a radiation therapy device includes the treatment head 406.

The treatment head 406 is a terminal execution device for performing radiation therapy. Merely by way of example, a proton treatment head, a linear accelerator treatment head, a gamma knife treatment head, or the like.

In some embodiments, the treatment head 406 is disposed on the image-guided system, and the calibration device 100 is installed on the treatment head 406 through the mounting holes 7.

The mechanical isocenter jig 404 is a calibration tool for calibrating a geometric reference of the radiation therapy device. Merely by way of example, a cruciform calibration frame installed on the treatment head 406, or a reference rod with a precision reference sphere or the like.

As shown in FIG. 13 and FIG. 14, the mechanical isocenter jig 404 includes a base 4041 and the central reference object 4042.

The base 4041 is a mechanical support structure for connecting the treatment head 406 and the central reference object 4042.

The base 4041 is installed on the treatment head 406 in the treatment room. Merely by way of example, the base 4041 is a cross frame structure or the like.

The central reference object 4042 is a reference component for calibrating the isocenter of the radiation therapy system in physical space. Merely by way of example, a precision calibration sphere, a crosshair target, a conical reference tip, or the like.

The central reference object 4042 is installed on the base 4041, specifically integrally formed with the base 4041.

The central reference object 4042 serves as the spatial physical representation of the isocenter position in the treatment system to guide configuration of the treatment system, i.e., when configuring the treatment system for performing radiation therapy in the treatment room, the treatment system is installed with the central reference object 4042 as the isocenter position, so that after installation of the treatment system is completed, a position of the central reference object 4042 is the isocenter position of the treatment system. The central reference object 4042 is installed at a center position of the base 4041, e.g., when the base 4041 adopts a cross frame structure, the central reference object 4042 is disposed at a cross intersection position of the cross frame structure.

In some implementations, the central reference object 4042 is a hemispherical structure, and a center of the hemispherical structure corresponds to the isocenter position of the treatment room. The loading hole 4033 is a hemispherical hole, and a diameter of the loading hole 4033 is the same as or similar to a diameter of the central reference object 4042.

An isocenter position of a treatment system is a reference point of a treatment device. Merely by way of example, the isocenter position of the treatment device is a spatial intersection point of a rotation axis of a gantry of a medical linear accelerator of the treatment device, a rotation axis of a treatment couch, and a rotation axis of a collimator during three-axis motion.

In some embodiments, an isocenter of an image-guided system coincides with the isocenter position of the treatment system.

Step S03: A laser beam verification phantom 403 is installed on the central reference object 4042, and the treatment head 406 is telescoped, and an isocenter position of the laser beam verification phantom 403 is moved to coincide with the isocenter position of the treatment system; wherein the laser beam verification phantom 403 includes three mutually perpendicular surfaces, each surface is preset with a positioning mark 4031 and a reflective section 4032 opposite to each laser lamp 402 of the plurality of laser lamps 402, and the positioning mark 4031 is adapted to a shape of a laser beam emitted by each laser lamp 402 of the plurality of laser lamps 402; the laser beam verification phantom 403 is provided with a loading hole 4033, a virtual connection line between the isocenter position of the laser beam verification phantom 403 and the loading hole 4033 extends to a center position of the central reference object 4042, and the virtual connection line between the isocenter position of the laser beam verification phantom 403 and the loading hole 4033 is configured to be coaxial with a telescopic path of the treatment head 406.

The laser beam verification phantom 403 is a calibration device 100 for verifying accuracy of a laser positioning system in a treatment room. For example, the laser beam verification phantom 403 is a cube with cross scale marks and a reflector, or a spherical verification phantom for detecting a laser intersection point, or the like. The laser beam verification phantom 403 has a cube structure, and the loading hole 4033 is formed by being recessed from one side surface of the laser beam verification phantom 403; at this time, a distance from the isocenter position of the laser beam verification phantom 403 to the center position of the central reference object 4042 is half of a side length of the laser beam verification phantom 403.

In some embodiments, the laser beam verification phantom 403 is installed on a mechanical isocenter jig 404.

The isocenter position of the laser beam verification phantom 403 is a reference point on the phantom for aligning with the isocenter position of the treatment system. As another example, the isocenter position of the laser beam verification phantom 403 is an intersection point of cross marks on three surfaces of a cube verification phantom, or a spherical center of a small metal sphere built inside the phantom, or the like.

The three mutually perpendicular surfaces of the laser beam verification phantom 403 are calibration planes on the phantom for respectively aligning with three groups of laser beams in the treatment room. For example, the three mutually perpendicular surfaces of the laser beam verification phantom 403 are a top surface and two adjacent side surfaces of a cube-shaped verification phantom, or the like.

In some embodiments, each surface is preset with the positioning mark 4031 and the reflective section 4032 opposite to each laser lamp 402 of the plurality of laser lamps 402.

In some embodiments, a quantity of surfaces for facing the laser lamps 402 is the same as a quantity of the laser lamps 402. For example, when the laser lamps 402 are three in count, the surfaces for facing the laser lamps 402 are also three in count, the three surfaces are mutually perpendicular, and the three surfaces correspond one-to-one to the three laser lamps 402.

The positioning mark 4031 is a reference identifier on a surface of the laser beam verification phantom 403 for aligning with a laser beam. As another example, the positioning mark 4031 is an etched cross scale line, a printed concentric ring, an embedded metal wire mark, or the like.

In some embodiments, the positioning mark 4031 is adapted to the shape of the laser beam emitted by the laser lamp 402. Specifically, a shape of the positioning mark 4031 is at least partially identical to a structure of a radial cross-sectional shape of the laser beam, so that the positioning mark 4031 coincides with the laser beam. When the positioning mark 4031 coincides with the laser beam, a center position of the positioning mark 4031 coincides with a center position of the laser beam. In addition, a connection line between the positioning mark 4031 and the isocenter position of the laser beam verification phantom 403 is perpendicular to a plane where the positioning mark 4031 is located. When the laser beam irradiates a corresponding surface of the laser beam verification phantom 403, the positioning mark 4031 serves as a reference mark for adjusting a position of the laser beam to cause the laser beam to coincide with the positioning mark 4031.

As shown in FIG. 13 and FIG. 14, the positioning mark 4031 is a part of the radial cross-sectional shape of the laser beam.

The reflective section 4032 is configured to verify whether the laser beam is perpendicular to the corresponding surface of the laser beam verification phantom 403. If the laser beam is not perpendicular to the corresponding surface of the laser beam verification phantom 403, and a connection line between a center position of the corresponding surface of the laser beam verification phantom 403 and the isocenter position of the laser beam verification phantom 403 does not coincide with an extension path of the laser beam, the laser beam cannot accurately extend to the isocenter position of the laser beam verification phantom 403, and an intersection point of a plurality of laser beams also does not coincide with the isocenter position of the treatment room. The corresponding surface of the laser beam verification phantom 403 is provided with the reflective section 4032, and the corresponding surface is configured to receive a corresponding laser beam.

The reflective section 4032 is configured to reflect the laser beam. If the reflective section 4032 and the positioning mark 4031 are located on a same surface of the laser beam verification phantom 403, the reflective section 4032 and the positioning mark 4031 have a common center. For example, a center position of the corresponding surface of the laser beam verification phantom 403 is the common center of the reflective section 4032 and the positioning mark 4031. When the laser beam is perpendicular to the corresponding surface of the laser beam verification phantom 403 and passes through the isocenter position of the laser beam verification phantom 403, the reflective section 4032 reflects the laser beam perpendicularly, and a reflection angle of the laser beam on the reflective section 4032 is 0°, so that a reflection path of the laser beam coincides with an original path of the laser beam. At this time, only one path of the laser beam emitted by one laser lamp 402 is observable, i.e., a reflection line of the laser beam is not visible. When the laser beam is not perpendicular but intersects the corresponding surface of the laser beam verification phantom 403 at a certain angle, the laser beam reflected by the reflective section 4032 deviates from the original path of the laser beam. At this time, for the laser beam emitted by one laser lamp 402, not only the emission path of the laser beam is observable, but also a reflection path exists, and a reflection line of the laser beam is visible. The reflection path of the laser beam does not coincide with the original path of the laser beam. Therefore, the laser beam needs to be adjusted to cause the laser beam to be perpendicular to the corresponding surface of the laser beam verification phantom 403. The reflective section 4032 is specifically a reflector, and the reflective section 4032 is disposed at the center position of the corresponding surface of the laser beam verification phantom 403.

The loading hole 4033 is a positioning interface on the laser beam verification phantom 403 for installing the laser beam verification phantom 403 to the mechanical isocenter jig 404. As another example, the loading hole 4033 is a precision cylindrical hole matching a positioning pin of the jig, or the like.

In some embodiments, the virtual connection line between the isocenter position of the laser beam verification phantom 403 and the loading hole 4033 extends to the center position of the central reference object 4042, and the virtual connection line between the isocenter position of the laser beam verification phantom 403 and the loading hole 4033 is configured to be coaxial with the telescopic path of the treatment head 406. Therefore, after the laser beam verification phantom 403 is installed on the central reference object 4042, the treatment head 406 is extended or retracted to move the isocenter position of the laser beam verification phantom 403 to an original position of the central reference object 4042, i.e., the isocenter position of the treatment system. A telescopic distance of the treatment head 406 is the distance from the isocenter position of the laser beam verification phantom 403 to the center position of the central reference object 4042.

In some embodiments, the loading hole 4033 of the laser beam verification phantom 403 is adapted to the central reference object 4042. After the laser beam verification phantom 403 is installed on the central reference object 4042, an electronic display level is used to measure a horizontality and a verticality of the laser beam verification phantom 403 relative to a wall or a floor of the treatment room, and a tolerance of the horizontality and the verticality of the laser beam verification phantom 403 is maintained within 0.01 mm. The treatment head 406 moves the isocenter position of the laser beam verification phantom 403 to the isocenter position of the treatment room by extending or retracting. The extending or retracting of the treatment head 406 is controlled by a handle. The handle is a component in the treatment system connected to the treatment head 406 and configured to control movement of the treatment head 406.

Step S04: Each laser lamp 402 is activated, so that each laser lamp 402 emits a laser beam (e.g., an X-ray, or the like) toward the laser beam verification phantom 403 along three mutually perpendicular directions, respectively.

The three mutually perpendicular directions are spatial reference directions in the treatment room for positioning a target region of a patient. As another example, the three mutually perpendicular directions are a left-right direction along a long axis of the treatment room, a front-rear direction perpendicular to the long axis, and an up-down direction perpendicular to the ground, or the like.

Step S05: Whether the laser beam emitted by each laser lamp 402 coincides with the positioning mark 4031 of the laser beam verification phantom 403 is determined; and whether a laser beam path reflected by the reflective section 4032 coincides with the laser beam path emitted by each laser lamp 402 is determined; if the laser beam emitted by each laser lamp 402 coincides with the positioning mark 4031 of the laser beam verification phantom 403; and a laser beam path reflected by the reflective section 4032 coincides with the laser beam path emitted by each laser lamp, a laser lamp position of each laser lamp 402 is not adjusted; if one of the laser beam emitted by each laser lamp 402 does not coincides with the positioning mark 4031 of the laser beam verification phantom 403; and a laser beam path reflected by the reflective section 4032 does not coincides with the laser beam path emitted by each laser lamp, the laser lamp position of each laser lamp 402 is adjusted, so that the laser beam emitted by the each laser lamp 402 coincides with the positioning mark 4031 of the laser beam verification phantom 403, and the laser beam path reflected by the reflective section coincides with the laser beam path emitted by each laser lamp 402.

In some embodiments, in the step S05, through visual observation, when a cross laser beam emitted by the laser lamp 402 does not coincide with the positioning mark 4031 of a cruciform structure, or a reflection path of the cross laser beam after being reflected by the reflective section 4032 does not coincide with an original path, the laser lamp position of the laser lamp 402 is determined to be not adjusted in place, and the laser lamp position of the laser lamp 402 is adjusted. When the cross laser beam emitted by the laser lamp 402 coincides with the positioning mark 4031 of the cruciform structure, and the reflection path of the cross laser beam after being reflected by the reflective section 4032 coincides with the original path, the laser lamp position of the laser lamp 402 is determined to be adjusted in place, and adjustment of the laser lamp 402 may be stopped.

In some embodiments, when the laser beam irradiates the corresponding surface of the laser beam verification phantom 403, the laser lamp position of the laser lamp 402 is adjusted to cause the laser beam emitted by the laser lamp 402 to coincide with the positioning mark 4031 on the corresponding surface of the laser beam verification phantom 403 and to be perpendicular to the corresponding surface of the laser beam verification phantom 403. The plurality of laser lamps 402 are adjusted by the above method. After the adjustment is completed, an intersection point of a plurality of laser beams emitted by the plurality of laser lamps 402 is the isocenter position of the laser beam verification phantom 403, i.e., the isocenter position of the treatment system.

Step S06: The mechanical isocenter jig and the laser beam verification phantom is removed, the laser lamps is turned on, and a projection position of the laser beam emitted by the laser lamps is marked.

In some embodiments, in the step S06, the projection position of the laser beam is a position where the laser beam is projected onto a wall or a floor of the treatment room when the laser beam is unobstructed. After the projection position of the laser beam is recorded, in a subsequent process, whether the projection position of the laser beam coincides with the recorded projection position may be observed, so that when the laser lamp 402 moves in the subsequent process, the movement may be quickly discovered according to a change in the projection position of the laser beam, and calibration may be performed to ensure that the laser beams emitted by the laser lamps intersect at the isocenter position of the treatment room.

Radiation therapy is one of three means for tumor treatment. A proton accelerator is a radiation therapy system that provides a proton beam for tumor treatment. The radiation therapy system is used to extract a proton beam with a prescribed dose and a three-dimensional dose distribution provided by a treatment planning system to a tumor target region of a patient to achieve a treatment purpose. In the radiation therapy system, after the proton beam is generated by an accelerator subsystem, the proton beam passes through an extraction channel, then is adjusted by a treatment head subsystem, and finally completes a beam extraction process. During treatment, to enable a proton beam to precisely reach a target region of a patient, the target region of the patient needs to be accurately moved to an isocenter position of a treatment room. When formulating a radiotherapy plan, a position of a tumor in CT imaging is roughly positioned via body surface markers to facilitate a coarse positioning of the patient before treatment. Before treating the patient in the treatment room, a coarse positioning of the patient is performed first. At this time, a laser lamp 402 for coarse positioning in the treatment room needs to be roughly aligned with the body surface markers of the patient, thereby completing the coarse positioning. A positioning system for coarse positioning in the treatment room includes a plurality of laser lamps 402 to generate a plurality of laser beams. The plurality of laser beams intersect at the isocenter position of the treatment room.

In some embodiments, by causing a laser beam to coincide with a positioning mark, and causing a beam path of the laser beam reflected by a reflective section 4032 to coincide with an original path, the laser beam is ensured to be perpendicular to a corresponding surface of a laser beam verification phantom and perpendicular to an isocenter plane where a treatment system is located. At this time, an intersection point of the plurality of laser beams is the isocenter position of the treatment system. Setting the positioning mark and the reflective section 4032 can relatively simply detect whether a position of the laser beam is accurate, facilitates calibration of the position of the laser beam, and effectively improves accuracy of a treatment positioning system in positioning the patient.

In some embodiments, a mechanical isocenter jig 404 is installed on a treatment head 406, wherein the mechanical isocenter jig 404 is preset with a central reference object 4042. More descriptions regarding the mechanical isocenter jig 404 and the central reference object 4042 may be found in the related description above.

In some embodiments, in the step S02, the plurality of laser lamps 402 are activated, and whether the laser beams emitted by the plurality of laser lamps 402 intersect at the central reference object 4042 of the mechanical isocenter jig 404 is observed.

In some embodiments, in the step S02, after the mechanical isocenter jig 404 is installed, the laser lamp 402 is activated, and a position of the laser lamp 402 is coarsely adjusted, so that the laser beams emitted by the plurality of laser lamps 402 intersect at the central reference object 4042 of the mechanical isocenter jig 404 under a visual state. The treatment head 406 is first rotated to a 180-degree position to facilitate installation of the mechanical isocenter jig 404. Adjustment of the position of the laser lamp 402 is achieved by fixedly installing the laser lamp 402 at different positions of a mounting groove 4011, thereby achieving flexible adjustment of the position of the laser lamp 402.

In some embodiments, adding a preliminary observation step for an intersection point of laser beams before fine calibration can quickly identify and eliminate significant installation deviations of the laser lamps.

In some embodiments, the positioning mark 4031 includes a horizontal scale line 4035 and a vertical scale line 4036. A virtual intersection point of the horizontal scale line and the vertical scale line is a center position of a corresponding surface of the laser beam verification phantom 403, and a virtual connection line of the horizontal scale line and the vertical scale line forms a cruciform structure, and the cruciform structure located on the same surface and the reflective section 4032 share a common center.

In some embodiments, as shown in FIG. 13 and FIG. 14, the positioning mark 4031 is a part of a radial cross-sectional shape of the laser beam. When the radial cross-sectional shape of the laser beam is a cruciform structure, the positioning mark 4031 includes a horizontal scale line 4035 and a vertical scale line 4036. The horizontal scale line 4035 and the vertical scale line 4036 are distributed on the upper, lower, left, and right sides of the center position of the positioning mark 4031. A connection line of the horizontal scale line 4035 and the vertical scale line 4036 forms a cruciform structure. The cruciform structure is the same as the radial cross-sectional shape of the laser beam. Furthermore, an intersection point of a virtual connection line of the horizontal scale line 4035 and the vertical scale line 4036 is a center position of the corresponding surface of the laser beam verification phantom 403, so that a connection line between the virtual intersection point and the isocenter position of the laser beam verification phantom 403 is perpendicular to the surface on which the positioning mark 4031 is set.

In some embodiments, by designing the positioning mark as a cruciform structure and aligning the cruciform structure with the center of the reflective section, a unified alignment reference is achieved during laser beam calibration. The unified alignment reference ensures that when the laser beam is aligned with the cruciform mark, a condition of self-collimation of the reflected optical path can be simultaneously satisfied, thereby completing synchronous calibration of the laser beam position and perpendicularity in one step, significantly improving calibration efficiency and accuracy.

In some embodiments, the loading hole 4033 is configured to install the laser beam verification phantom on the central reference object 4042. The loading hole 4033 has an identical structure to at least a portion of the central reference object 4042 of the mechanical isocenter jig 404. A wall forming the loading hole 4033 fits an outer wall of the central reference object 4042, so that the laser beam verification phantom 403 remains fixed relative to the central reference object 4042, thereby enabling accurate installation of the laser beam verification phantom 403 on the central reference object 4042.

In some embodiments, by matching the structure of the loading hole of the laser beam verification phantom with the structure of the central reference object of the mechanical isocenter jig, rapid and precise positioning and installation of the phantom at the isocenter point is achieved.

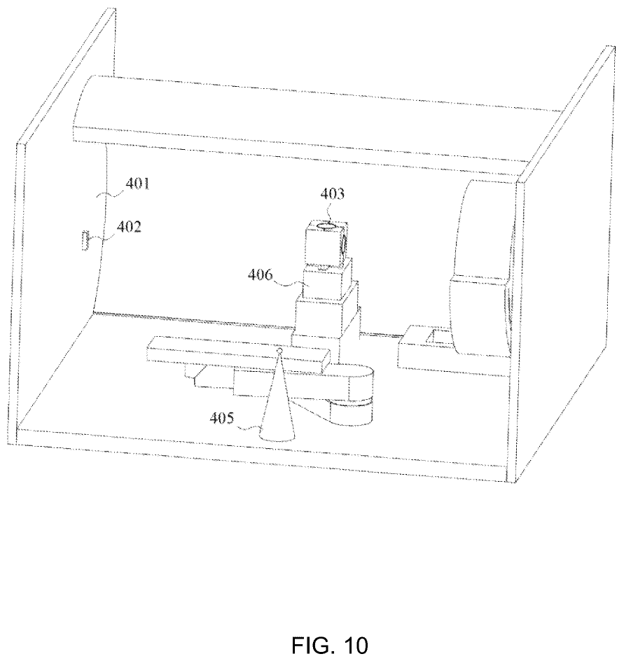

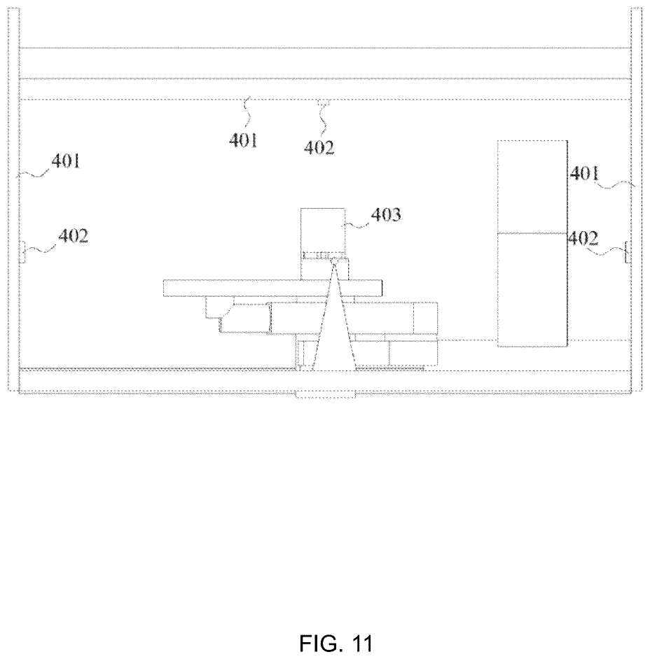

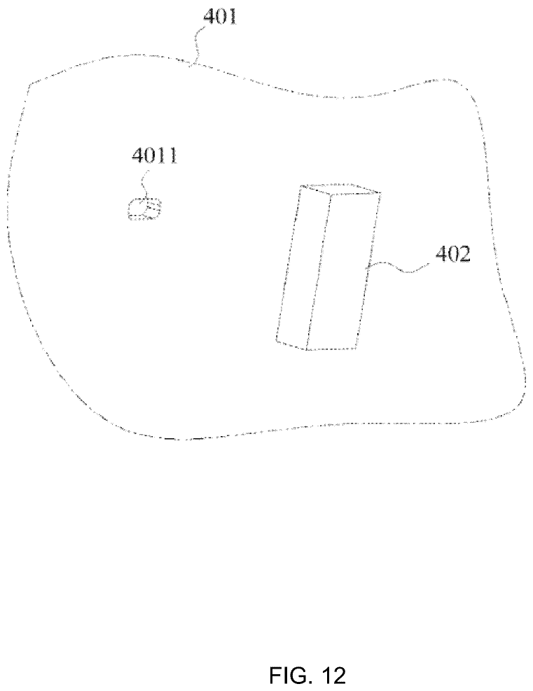

FIG. 10 is a schematic structural diagram of a treatment room positioning system installed in a treatment room according to some embodiments of the present disclosure. FIG. 11 is a schematic structural diagram of another perspective when a treatment room positioning system is installed in a treatment room according to some embodiments of the present disclosure. FIG. 12 is a partial exploded view of a wall body and a laser lamp in a treatment room positioning system according to some embodiments of the present disclosure.

In some embodiments, as shown in FIG. 10, a treatment room positioning system includes a wall body 401, preset with a mounting position; the plurality of laser lamps 402, installed at the mounting position of the wall body 401, and the laser beam verification phantom 403, wherein the isocenter position of the laser beam verification phantom is located at an intersection point of laser beams emitted by the plurality of laser lamps 402.

The wall body 401 is a building structure in the treatment room for installing positioning equipment. For example, the wall body 401 may be a side wall, a ceiling, or a floor of the treatment room, or the like.

In some embodiments, the mounting position is preset on the wall body 401. More descriptions regarding the mounting position may be found in the related description above.

The laser lamp 402 is an emission device for emitting a laser beam. For example, the laser lamp 402 is an emission device for emitting a cross laser beam. The cross laser beam is a laser beam whose radial cross-section is a crossed cruciform structure. A center position of the laser beam being an intersection point of the cruciform structure can better achieve positioning of the isocenter position. Center positions of laser beams emitted by the plurality of laser lamps 402 intersect at the isocenter position of the treatment system.

As described in FIG. 11 and FIG. 12, both the wall body 401 and the laser lamp 402 include a plurality. The quantity of the wall body 401 is the same as that of the laser lamp 402. Each wall body 401 is preset with the mounting position. Each mounting position is configured to install one laser lamp 402.

In some embodiments, the quantities of the wall body 401, the laser lamp 402, and marks preset according to a tumor position of the patient are all three. The marks preset according to the tumor position of the patient are located on the left and right sides and the upper side of the isocenter point position of the tumor. Correspondingly, the wall body 401 consists of the left and right walls and the ceiling of the treatment room, so that the laser lamps 402 installed on the wall body 401 correspond one-to-one with the preset marks. When the isocenter point position of the patient's tumor coincides with the isocenter position of the treatment system, the patient reaches a treatment position. Afterwards, the treatment system can be controlled to perform radiotherapy on the patient.

In some embodiments, the laser lamp 402 is installed on the wall body 401 via the mounting groove 4011.

In some embodiments, to enable adjustment of the laser lamp 402 after it is installed on the wall body 401, the mounting groove 4011 is an elongated slot. An extension direction of the elongated slot is consistent with a direction in which the laser lamp 402 needs to be adjusted. For example, after the laser lamp 402 is installed on the wall body 401, if the laser lamp 402 needs to be adjusted along a horizontal direction, the extension direction of the elongated slot extends along the horizontal direction. At this time, the laser lamp 402 can be fixedly installed at a plurality of positions of the elongated slot along the horizontal direction to achieve adjustment of the position of the laser lamp 402. Furthermore, the mounting groove 4011 is set as a cross-shaped slot or a slot of another structure as needed, so that the laser lamp 402 is located at a plurality of positions of the mounting groove 4011 along a plane, thereby enabling adjustment of the position of the laser lamp 402 in horizontal and vertical directions.

In some embodiments, by integrating precisely calibrated laser lamps and a verification phantom, a stable spatial coordinate system based on the isocenter point is constructed in the treatment room. The stable spatial coordinate system provides medical staff with an intuitive and reliable body surface positioning reference, thereby ensuring that the target region of the patient can be quickly and accurately positioned to the focus of a treatment radiation beam before each treatment, ultimately significantly improving the accuracy and repeatability of radiotherapy.

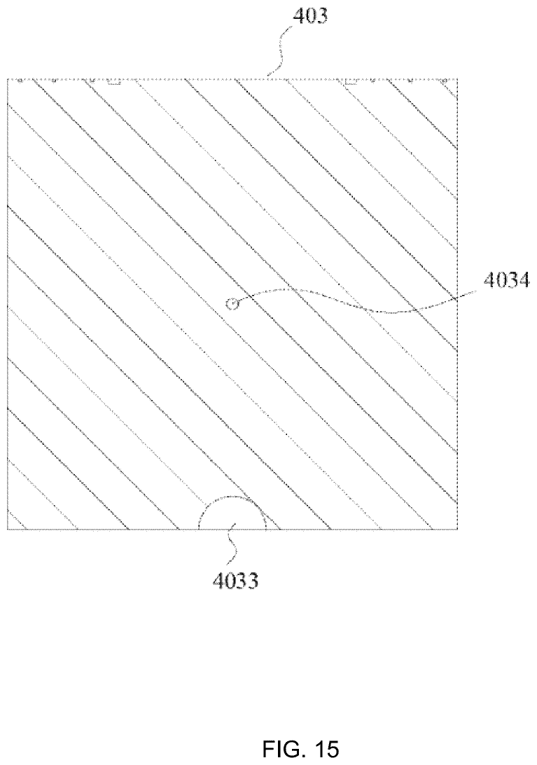

FIG. 15 is a cross-sectional view of the laser beam verification phantom according to some embodiments of the present disclosure.

As shown in FIG. 15, in some embodiments, the laser beam verification phantom 403 is provided with a positioning metal sphere 4034. A spherical center of the positioning metal sphere 4034 coincides with the isocenter position of the laser beam verification phantom 403. The positioning metal sphere 4034 is configured as a physical representation of the isocenter position of the laser beam verification phantom 403.

In some embodiments, when the isocenter position of the laser beam verification phantom 403 coincides with the isocenter position of the treatment system, the positioning metal sphere 4034 serves as a physical representation of the isocenter position of the treatment system.

In some embodiments, after position adjustment of the treatment system is completed, whether components in the treatment system have moved needs to be detected periodically. For example, the detection is performed once a month. At this time, the positioning metal sphere 4034 serves as the physical representation of the isocenter position of the treatment system. The positioning metal sphere 4034 is irradiated by an X-ray of the treatment system to form a projection. A position of the positioning metal sphere 4034 in the formed projection image is observed. If the position of the positioning metal sphere 4034 is at a center of the projection image, the position of the treatment system is determined to be stationary. If the position of the positioning metal sphere 4034 is not at the center of the projection image, the position of the treatment system is determined to have been displaced, and positions of various components in the treatment system need to be recalibrated.

In some embodiments, by setting the positioning metal sphere coinciding with the isocenter point of the phantom, a stable, precise, and clearly visible physical reference point is provided for the treatment system in imaging. The physical reference point also facilitates rapid verification of the isocenter position of the treatment system during periodic inspections, effectively ensuring long-term reliability of geometric accuracy in radiotherapy.

What is claimed is:

1. A calibration device for an image-guided system, comprising:
at least two optical path channels, wherein each of the at least two optical path channels penetrates the calibration device, and the at least two optical path channels are configured to allow rays to pass through; the at least two optical path channels intersect with each other, and an intersection point of the at least two optical path channels is a center point of the calibration device;
at least two imaging assemblies, wherein each of the at least two imaging assemblies includes two imaging surfaces, and the two imaging surfaces are disposed at two ends of a same optical channel among the at least two optical path channels; each of the two imaging surfaces is provided with a marking portion, the marking portion is provided with a central marking point, and the central marking point is located in an extending direction of the same one of the at least two optical path channels; a connection line between the central marking points of the two imaging surfaces in each of the at least two imaging assemblies passes through the center point of the calibration device.

2. The calibration device of claim 1, wherein the at least two optical path channels are formed by extending along a pair of intersecting diagonals in the calibration device; and/or, the two imaging surfaces are disposed at a vertex of the calibration device.

3. The calibration device of claim 1, wherein the calibration device is provided with a recessed groove and a plurality of light-transmitting holes, the center point of the calibration device is located in the recessed groove, one end of each of a plurality of light-transmitting holes penetrates the calibration device, and another end of each of the plurality of light-transmitting holes communicates with the recessed groove; a pair of light-transmitting holes located on opposite sides of the center point of the calibration device and the recessed groove form one of the at least two optical path channels together; and
the two imaging surfaces block the plurality of light-transmitting holes.

4. The calibration device of claim 1, further comprising:
a calibration sphere, configured to indicate an isocenter of the image-guided system, wherein the calibration sphere coincides with the center point of the calibration device;
a mounting hole, configured to fix the calibration device to the image-guided system;
a calibration hole, configured to install the calibration sphere;
a positioning channel, penetrating the calibration device, wherein the positioning channel is configured to allow light to pass through; the positioning channel extends along a center line in a horizontal direction of the calibration device, and the positioning channel passes through the center point of the calibration device; and
a positioning assembly, including a pair of positioning surfaces, wherein the pair of positioning surfaces are disposed at two opposite ends of the positioning channel, the pair of positioning surfaces are provided with positioning portions, the positioning portions are provided with central positioning points, and a connection line between the central positioning points of the pair of positioning surfaces passes through the center point of the calibration device.

5. The calibration device of claim 4, wherein the calibration device is provided with a recessed groove and a pair of light-transmitting holes, the center point of the calibration device is accommodated in the recessed groove, one end of each of the pair of light-transmitting holes penetrates the calibration device, and another end of each of the pair of light-transmitting holes communicates with the recessed groove; the pair of light-transmitting holes and the recessed groove form the positioning channel together; the pair of positioning surfaces block the pair of light-transmitting holes; the positioning channel intersects with one of the at least two optical path channels;
and/or, a shape of the positioning portion is a crosshair structure, and an intersection point of the crosshair is the central positioning point.

6. An image-guided system, comprising:
the calibration device of claim 1;
at least one pair of ray generating devices, configured to generate the rays passing through the at least two optical path channels of the calibration device, wherein rays generated by the at least one pair of ray generating devices intersect with each other;
at least one pair of image processing devices, configured to receive the rays passing through the at least two optical path channels of the calibration device and generate image information.

7. The image-guided system of claim 6, further comprising a control device, wherein the control device is configured to control movement of at least one of the at least one pair of ray generating devices or the at least one pair of image processing devices;
and/or, the at least one pair of ray generating devices are X-ray tubes, and the at least one pair of image processing devices are flat panel detectors.

8. A position calibration method of the image-guided system of claim 6, comprising:
installing the calibration device to the image-guided system, and aligning the center point of the calibration device with a target point of the image-guided system;
emitting, by the at least one pair of ray generating devices, the rays, wherein the rays pass through the calibration device and are received by the at least one pair of image processing devices, and the at least one pair of image processing devices generate the image information of the received rays;
transmitting, by the at least one pair of image processing devices, the image information to a data processing device, and obtaining, by the data processing device, positional offset information of at least one of the at least one pair of ray generating devices or the at least one pair of image processing devices based on the image information;
adjusting a position of at least one of the at least one pair of ray generating devices or the at least one pair of image processing devices based on the positional offset information;
until the position of at least one of the at least one pair of ray generating devices or the at least one pair of image processing devices is adjusted to a designated position.

9. The method for position calibration of claim 8, wherein the emitting, by the at least one pair of ray generating devices, the rays, wherein the rays pass through the calibration device and are received by the at least one pair of image processing devices, and the at least one pair of image processing devices generate the image information of the received rays includes:
the rays passing through the two imaging surfaces of the calibration device;
wherein the at least one pair of image processing devices corresponds one-to-one with the at least one pair of ray generating devices, each image processing device receives rays generated by one of the at least one pair of ray generating devices corresponding to each image processing device, and generates the image information including two imaging surface projections.

10. The method for position calibration of claim 9, wherein the transmitting, by the at least one pair of image processing devices, the image information to a data processing device, and obtaining, by the data processing device, positional offset information of at least one of the at least one pair of ray generating devices or the at least one pair of image processing devices based on the image information includes:
designating a center point of an image generated by the at least one pair of image processing devices as a first origin point, obtaining a coordinate point A of a central marking point of one of the two imaging surface projections in the image information that is close to the target point of the image-guided system as (X1, Y1), and obtaining a coordinate point B of a central marking point of one of the two imaging surface projections in the image information that is far from the target point of the image-guided system as (X2, Y2);
wherein a positional offset of the at least one pair of ray generating devices in an X axis direction is (X1+X2)/2, and a positional offset of the at least one pair of ray generating devices in a Y axis direction is (Y1+Y2)/2;
the adjusting a position of at least one of the at least one pair of ray generating devices or the at least one pair of image processing devices based on the positional offset information includes:
in response to X1>X2, adjusting the at least one pair of ray generating devices to move a distance of (X1+X2)/2 along a negative direction of an X axis; in response to X1<X2, adjusting the at least one pair of ray generating devices to move a distance of (X1+X2)/2 along a positive direction of the X axis; and
in response to Y1>Y2, adjusting the at least one pair of ray generating devices to move a distance of (Y1+Y2)/2 along a negative direction of an Y axis; in response to Y1<Y2, adjusting the at least one pair of ray generating devices to move a distance of (Y1+Y2)/2 along a positive direction of the Y axis.

11. The method for position calibration of claim 10, wherein the until the position of at least one of the at least one pair of ray generating devices or the at least one pair of image processing devices is adjusted to a designated position includes:
adjusting the at least one pair of ray generating devices to a designated position until the positional offset of the at least one pair of ray generating devices in the X axis direction and the positional offset in the Y axis direction are both less than a specified threshold.

12. The method for position calibration of claim 9, wherein the transmitting, by the at least one pair of image processing devices, the image information to a data processing device, and obtaining, by the data processing device, positional offset information of at least one of the at least one pair of ray generating devices or the at least one pair of image processing devices based on the image information includes:
designating a central marking point of one of the imaging surface projections in an image generated by the at least one pair of image processing devices as a second origin point, obtaining a coordinate of a first endpoint C of the target point in the image-guided system, wherein the first endpoint C is adjacent to the marking portion within the one of the imaging surface projection, as (X3, Y3), and obtaining a coordinate of a second endpoint D of the target point in the image-guided system, wherein the first endpoint C is far from the marking portion within the one of the imaging surface projection, as (X4, Y4); a ratio of a distance between a first marking point in the marking portion that is formed by a projection of the first endpoint C and the central marking point to a distance between a second marking point in the marking portion that is formed by a projection of the second endpoint D and the central marking point is a:b;
in response to X3>X4, and a ratio of a distance between the first endpoint C and the second origin point to a distance between the second endpoint D and the second origin point being greater than a:b, determining that an image processing device including the second origin point is offset clockwise;
in response to X3>X4, and the ratio of the distance between the first endpoint C and the second origin point to the distance between the second endpoint D and the second origin point being less than a:b, determining that the image processing device including the second origin point is offset counterclockwise;
in response to X3<X4, and the ratio of the distance between the first endpoint C and the second origin point to the distance between the second endpoint D and the second origin point being greater than a:b, determining that the image processing device including the second origin point is offset counterclockwise; in response to X3<X4, and the ratio of the distance between the first endpoint C and the second origin point to the distance between the second endpoint D and the second origin point being less than a:b, determining that the image processing device including the second origin point is offset clockwise;
the adjusting a position of at least one of the at least one pair of ray generating devices or the at least one pair of image processing devices based on the positional offset information includes:
in response to the image processing device including the second origin point being offset clockwise, rotating the image processing device including the second origin point counterclockwise by a rated angle about a rotation axis;
in response to the image processing device including the second origin point being offset counterclockwise, rotating the image processing device including the second origin point clockwise by the rated angle about the rotation axis;
wherein the rotation axis is a straight line perpendicular to a connection line between the first endpoint C and the second endpoint D and passing through the second origin point.

13. The method for position calibration of claim 12, wherein the until the position of at least one of the at least one pair of ray generating devices or the at least one pair of image processing devices is adjusted to a designated position includes:
after the image processing device including the second origin is rotated by the rated angle, an offset direction of the image processing device including the second origin changing, adjusting the image processing device including the second origin point to a designated position.

14. The method for position calibration of claim 8, wherein the calibration device further comprises:
a positioning channel, penetrating through the calibration device, wherein the positioning channel is configured to allow light to pass through; the positioning channel extends along a center line of the calibration device, and the positioning channel passes through a center point of the calibration device;
a positioning assembly, including a pair of positioning surfaces, wherein the pair of positioning surfaces is disposed at opposite ends of the positioning channel, the pair of positioning surfaces is provided with positioning portions, the positioning portions are provided with central positioning points, and a connection line of the pair of central positioning points of the pair of positioning surfaces passes through the center point of the calibration device;
the installing the calibration device to the image-guided system, and aligning the center point of the calibration device with a target point of the image-guided system includes:
using a survey tool to form a horizontal optical path passing through the target point of the image-guided system, installing the calibration device on the image-guided system, and making the pair of central positioning points of the calibration device coincide with each other along a projection of the horizontal optical path of the survey tool;
rotating the calibration device, so that rays generated by the at least one pair of ray generating devices pass through at least two imaging surfaces of the calibration device.

15. A calibration method for a treatment room positioning system, wherein the treatment room positioning system is configured to assist in positioning a target region in a treatment system, wherein the calibration method comprises:
installing a plurality of laser lamps in the treatment room;
installing a mechanical isocenter jig on a treatment head, wherein the mechanical isocenter jig is preset with a central reference object, and the central reference object is configured as a spatial physical representation of an isocenter position of the treatment system to configure the treatment system;
installing a laser beam verification phantom on the central reference object, telescoping the treatment head, and moving an isocenter position of the laser beam verification phantom to coincide with the isocenter position of the treatment system; wherein the laser beam verification phantom includes three mutually perpendicular surfaces, each surface is preset with a positioning mark and a reflective section opposite to each laser lamp of the plurality of laser lamps, and the positioning mark is adapted to a shape of a laser beam emitted by each laser lamp; the laser beam verification phantom is provided with a loading hole, a virtual connection line between the isocenter position of the laser beam verification phantom and the loading hole extends to a central position of the central reference object, and the virtual connection line between the isocenter position of the laser beam verification phantom and the loading hole is configured to be coaxial with a telescopic path of the treatment head;
activating each laser lamp, so that each laser lamp emits a laser beam toward the laser beam verification phantom along three mutually perpendicular directions, respectively;
determining whether the laser beam emitted by each laser lamp coincides with the positioning mark of the laser beam verification phantom; and whether a laser beam path reflected by the reflective section coincides with the laser beam path emitted by each laser lamp; if the laser beam emitted by each laser lamp coincides with the positioning mark of the laser beam verification phantom; and a laser beam path reflected by the reflective section coincides with the laser beam path emitted by each laser lamp, not adjusting a laser lamp position of the each laser lamp; if one of the laser beam emitted by each laser lamp does not coincides with the positioning mark of the laser beam verification phantom; and a laser beam path reflected by the reflective section does not coincides with the laser beam path emitted by each laser lamp, adjusting the laser lamp position of each laser lamp, so that the laser beam emitted by the each laser lamp coincides with the positioning mark of the laser beam verification phantom, and the laser beam path reflected by the reflective section coincides with the laser beam path emitted by each laser lamp.

16. The calibration method of claim 15, wherein the installing a mechanical isocenter jig on a treatment head, wherein the mechanical isocenter jig is preset with a central reference object, and the central reference object is configured as a spatial physical representation of an isocenter position of the treatment system to configure the treatment system includes:
activating the plurality of laser lamps, and observing whether the laser beams emitted by the plurality of laser lamps intersect at the central reference object of the mechanical isocenter jig.

17. The calibration method of claim 15, wherein the positioning mark includes a horizontal scale line and a vertical scale line, a virtual intersection point of the horizontal scale line and the vertical scale line is a central position of a corresponding surface of the laser beam verification phantom, and a virtual connection line of the horizontal scale line and the vertical scale line forms a cruciform structure, and the cruciform structure located on the same surface and the reflective section share a common center.

18. The calibration method of claim 17, wherein the laser beam verification phantom is provided with the loading hole, the loading hole has an identical structure to at least a portion of the central reference object of the mechanical isocenter jig, and the loading hole is configured to install the laser beam verification phantom on the central reference object.

19. A treatment room positioning system, wherein the treatment room positioning system is configured to execute the calibration method of claim 15; the treatment room positioning system comprises:
a wall body, preset with a mounting position;
the plurality of laser lamps, installed at the mounting position of the wall body; and
the laser beam verification phantom, wherein the isocenter position of the laser beam verification phantom is located at an intersection point of laser beams emitted by the plurality of laser lamps.

20. The treatment room positioning system of claim 19, wherein the laser beam verification phantom is provided with a positioning metal sphere, a spherical center of the positioning metal sphere coincides with the isocenter position of the laser beam verification phantom, and the positioning metal sphere is configured as a physical representation of the isocenter position of the laser beam verification phantom.
